(19) United States
(12) Patent Application Publication
(10) Pub. No.: US 2026/0366975 A1
(43) Pub. Date: Nov. 19, 2026

(54) MULTI-CHANNEL HEMATOLOGY FLOW SYSTEM

(71) Applicant: Beckman Coulter, Inc., Brea, CA (US)

(72) Inventors: Bart WANDERS, Trabuco Canyon, CA (US); John ROCHE, Brisbane, CA (US); Ken GOOD, Glendale, CA (US); Carol QUON, Ventura County, CA (US); Linda GARLAUS, Davie, FL (US); Rigoberto ROCHE, Orlando, FL (US)

(21) Appl. No.: 19/136,268

(22) PCT Filed: Dec. 5, 2023
(86) PCT No.: PCT/US2023/082526

§ 371 Date: Jun. 5, 2025

Related U.S. Application Data
(60) Provisional application No. 63/430,233, filed on Dec. 5, 2022.
(60) Provisional application No. 63/430,232, filed on Dec. 5, 2022.
(60) Provisional application No. 63/430,237, filed on Dec. 5, 2022.

(51) Int. Cl. G01N 15/01; G01N 15/1429; G16B 45/00

(52) U.S. Cl. CPC G01N 15/01; G01N 15/1429; G16B 45/00; G01N 2015/012; G01N 2015/018

(57) ABSTRACT

A sample analysis system may comprise a flow imaging module, an alternative system, a fluidics system, and an analysis module. In such a system, the flow imaging module may comprise a flowcell and an image capture device, and the alternative system may comprise an impedance detector. The fluidics system may be adapted to flow portions of samples through the flow imaging and alternative systems. The analysis module may be adapted to determine values for a first plurality of parameters using data from the flow imaging module, and values for a second plurality of parameters using data from the alternative system.

### CROSS REFERENCE TO RELATED APPLICATIONS

This claims priority from, and is a nonprovisional of, provisional patent application 63/430,233, entitled “Multi-Channel Hematology Flow System” and filed in the U.S. patent and trademark office Dec. 5, 2022. That application is hereby incorporated by reference in its entirety.

### BACKGROUND

Blood cell analysis is one of the most commonly performed medical tests for providing an overview of a patient's health status. A blood sample can be drawn from a patient's body and stored in a test tube containing an anticoagulant to prevent clotting. A whole blood sample normally comprises three major classes of blood cells including red blood cells (erythrocytes), white blood cells (leukocytes) and platelets (thrombocytes). Each class can be further divided into subclasses of members. For example, five major types or subclasses of white blood cells (WBCs) have different shapes and functions. White blood cells may include neutrophils, lymphocytes, monocytes, eosinophils, and basophils. There are also subclasses of the red blood cell types. The appearances of particles in a sample may differ according to pathological conditions, cell maturity and other causes. Red blood cell subclasses may include reticulocytes and nucleated red blood cells.

Traditional blood cell analysis techniques have utilized principles such as impedance or Coulter principle, and fluorescence or light scatter in order to count and measure cells. These techniques utilize indirect measurements and therefore may be limited in the amount and quality of information that can be provided. Additionally, slide review is a common secondary step where a test result will need further analysis (e.g., to confirm a result, or to assess some abnormality), which is typically done through an automated or manual slide imaging step.

There is a need for improvements to the traditional blood cell analysis techniques which leverage new techniques to optimize workflow and improve cell analysis to improve patient outcomes.

### SUMMARY

Described herein are devices, systems and methods for classifying objects such as cells using analyzers, such as a biological analyzer/biological analysis system which captures cell images. In some embodiments, both images and additional values of blood cells from a blood sample (e.g., impedance-derived values, volume-conductivity-scatter-derived values, fluorescence-derived values, and/or spectrophotometry-derived values) may be used in such classification or other types of analysis. In some embodiments, images, image-derived values, and values derived from non-imaging techniques are presented on a user interface (e.g., screen).

In some embodiments, cell information obtained from images and cell information obtained through non-imaging techniques (e.g., impedance, fluorescence, or spectrophotometry) may overlap, for example where imaging is used to obtain a first parameter of a first particle (e.g., red blood cell count, or platelet count) and non-imaging is also used to obtain the parameter (e.g., red blood cell count, or platelet count). In some embodiments, both values are presented on a user interface.

In some embodiments, there may be provided a sample analysis system comprising a fluidics system and one or more processors. In such a system, the fluidics system may be adapted to flow a first portion of a blood sample through a first module, the first module being a flow imaging module comprising a flowcell and an image capture device configured to capture a plurality of images of cells of the first portion of the blood sample. The fluidics system may also be adapted to flow a second portion of the blood sample through a second module, the second module configured to test for one or more numerical parameters of cells of the second portion of the blood sample. The one or more processors may be programmed to perform a set of acts. These acts may comprise determining the one or more numerical parameters of cells of the second portion of the blood sample, and presenting a computing interface comprising the plurality of images of the cells of the first portion of the blood sample and the one or more numerical parameters of the cells of the second portion of the blood sample. Corresponding methods and computer readable media may also be implemented based on this disclosure. Accordingly, a system such as described should be understood as being illustrative only, and should not be treated as imposing limitations on the protection provided by this document or any related document.

In some embodiments, an imaging system utilizes an image analysis algorithm in order to analyze cell images and report particular information about the cell-such as cell type, cell count, or other quantitative information about the cell. The algorithm can utilize, for example a trained machine learning algorithm, or pixel analysis in order to analyze images.

In some embodiments, a biological analysis system provides a review indication (e.g., flag) associated with an analyzed biological sample. For instance, a review indication can be associated with any of the following-reported count of a particular cell type, abnormal result, abnormal cell type

In some embodiments, a biological analysis system of method includes image review on a user interface where a user can confirm a sample result through the user interface image review. In some embodiments, a biological analysis method includes analyzing a biological sample, presenting images of cells of the biological sample on a user interface, and confirming a sample result through the user interface image review. In some embodiments, the user interface image review includes a review indication (e.g., flag) associated with an analyzed biological sample.

In some embodiments, a multi-channel analyzer or multi-channel analysis system comprises an imaging channel or module, and one or more non-imaging channels. The one or more non-imaging channels utilize any of, for example, impedance, volume-conductivity-scatter, fluorescence, or spectrophotometry.

In some embodiments, methods of the embodiments described above and herein are contemplated.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 is a schematic illustration, partly in section and not to scale, showing operational aspects of an exemplary flowcell, autofocus system and high optical resolution imaging device for sample image analysis using digital image processing.

FIG. 1A shows an optical bench arrangement according to various embodiments.

FIG. 1B shows another optical bench arrangement according to various embodiments.

FIG. 1C is a block diagram of a hematology analyzer according to various embodiments.

FIG. 2 schematically depicts aspects of a cellular analysis system, according to various embodiments.

FIG. 3 provides a system block diagram illustrating aspects of a cellular analysis system according to various embodiments.

FIG. 4 illustrates aspects of an automated cellular analysis system for evaluating the white blood cell status of an individual, according to embodiments of the present invention.

FIG. 5 illustrates a process for deriving data from captured images and measured impedance according to various embodiments.

FIG. 6 illustrates a process for reviewing derived data from captured images according to various embodiments.

FIG. 7 provides an example user interface according to various embodiments.

FIG. 8 illustrates another process for deriving data from captured images and measured impedance according to various embodiments.

FIG. 9 illustrates a module system which may be utilized in some implementations of the disclosed technology.

FIG. 10 illustrates a perspective view of an illustrative optical system of a fluorescence analyzer;

FIG. 11 illustrates a process which may be used to stain a sample;

FIG. 12 illustrates a system block diagram illustrating aspects of a cellular analysis system according to embodiments of the present invention.

FIG. 13 illustrates a spectrophotometry system which be utilized in some implementations of the disclosed technology.

FIG. 14 illustrates a method of use of the spectrophotometry system of FIG. 13.

FIG. 15 illustrates a dual channel testing apparatus having an imaging system and a non-imaging system;

FIG. 16 illustrates schematic view of the non-imaging system of FIG. 15;

FIG. 17 illustrates the imaging system of FIG. 15;

FIG. 18 illustrates an architecture for a machine learning model which can be used in analyzing images; and

FIG. 19 is an example of a stage such as might be included in a machine learning model following the architecture of FIG. 18.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The present disclosure relates to apparatus, systems, compositions, and methods for analyzing a sample containing particles. One embodiment may include an automated particle imaging system which comprises an analyzer which may be, for example, a visual analyzer. In some embodiments, the visual analyzer may further comprise a processor to facilitate automated analysis of the images.

Additional embodiments can include other particle analysis systems along with a visual analyzer. These other particle analysis systems can comprise, for instance, automated impedance measurement systems, fluorescence measurement systems, spectrophotometry measurement systems, conductivity systems, light scatter systems, additional imaging systems, or other types of systems which may be used to gather data regarding a sample. In some embodiments, the analyzer may further comprise a processor to facilitate automated analysis of the images and/or to present one or more interfaces which could present data from multiple channels (e.g., an interface which could present data derived from images captured by an imaging device, as well as data derived from measurements made by one or more of an impedance, conductivity, light scatter, fluorescence, or spectrophotometry system). In some embodiments, a biological analyzer or biological analysis system comprises multiple channels or modules-including an imaging channel/module and one or more non-imaging channel/modules (e.g., impedance, conductivity, scatter, fluorescence, spectrophotometry).

### Imaging System

According to some aspects of this disclosure, a system comprising a visual/imaging analyzer or module may be provided for obtaining images of a sample comprising particles suspended in a liquid. Such a system may be useful, for example, in characterizing particles in biological fluids, such as detecting and quantifying erythrocytes, reticulocytes, nucleated red blood cells, platelets, and white blood cells, including white blood cell differential counting, categorization and subcategorization and analysis. Other similar uses such as characterizing blood cells from other fluids are also contemplated.

The discrimination and/or classification of blood cells in a blood sample is an exemplary application for which the subject matter is particularly well suited, though other types of body fluid samples may be used. For example, aspects of the disclosed technology may be used in analysis of a non-blood body fluid sample comprising blood cells (e.g., white blood cells and/or red blood cells), such as serum, bone marrow, lavage fluid, effusions, exudates, cerebrospinal fluid, pleural fluid, peritoneal fluid, and amniotic fluid. It is also possible that the sample can be a solid tissue sample (e.g., a biopsy sample that has been treated to produce a cell suspension). The sample may also be a suspension obtained from treating a fecal sample, or a urine sample. A sample may also be a laboratory or production line sample comprising particles, such as a cell culture sample. The term sample may be used to refer to a sample obtained from a patient or laboratory or any fraction, portion or aliquot thereof. The sample can be diluted, divided into portions, or stained in some processes.

In some aspects, samples are presented, imaged and analyzed in an automated manner. In the case of blood samples, the sample may be substantially diluted with a suitable diluent or saline solution, which reduces the extent to which the view of some cells might be hidden by other cells in an undiluted or less-diluted sample. The cells can be treated with agents that enhance the contrast of some cell aspects, for example using permeabilizing agents to render cell membranes permeable, and histological stains to adhere in and to reveal features, such as granules and the nucleus. In some cases, it may be desirable to stain an aliquot of the sample for counting and characterizing particles which include reticulocytes, nucleated red blood cells, and platelets, and for white blood cell differential, characterization and analysis. In other cases, samples containing red blood cells may be diluted before introduction to the flow cell and/or imaging in the flow cell or otherwise.

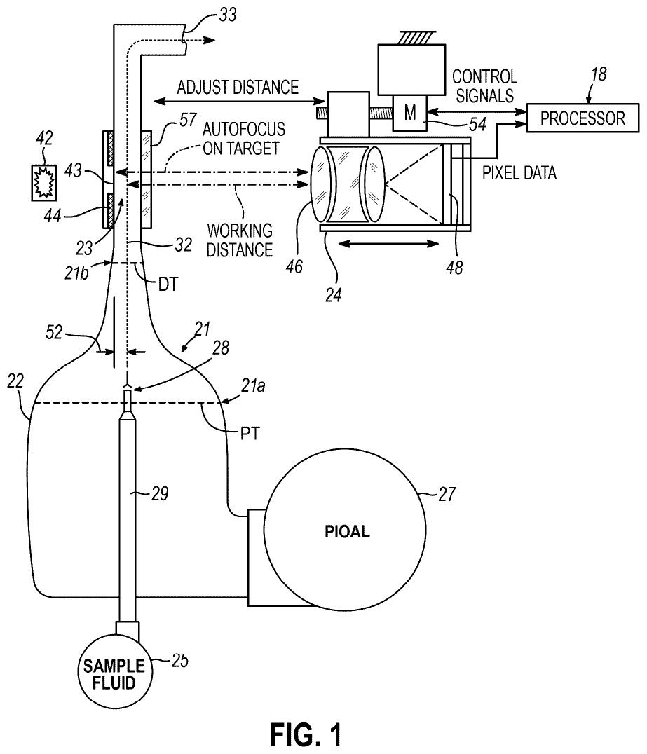

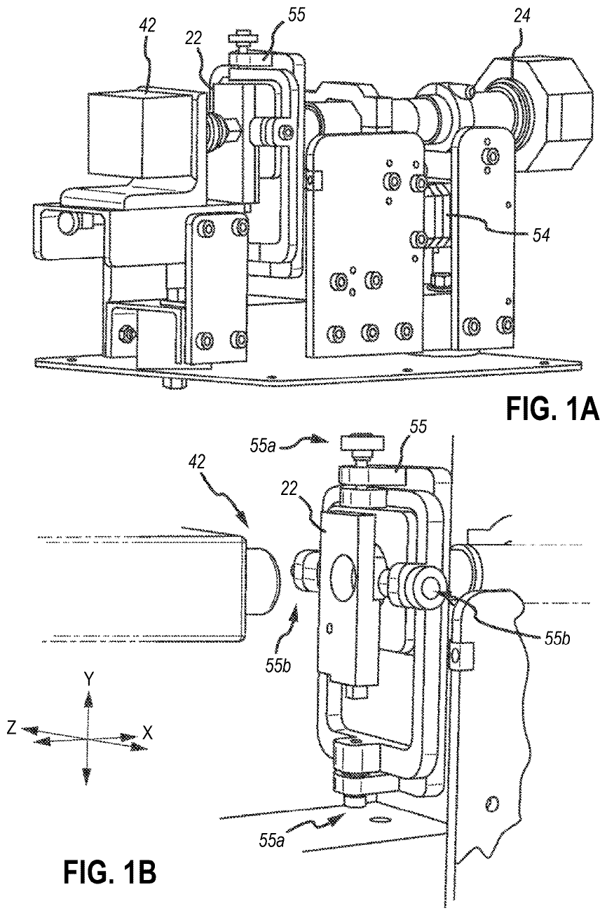

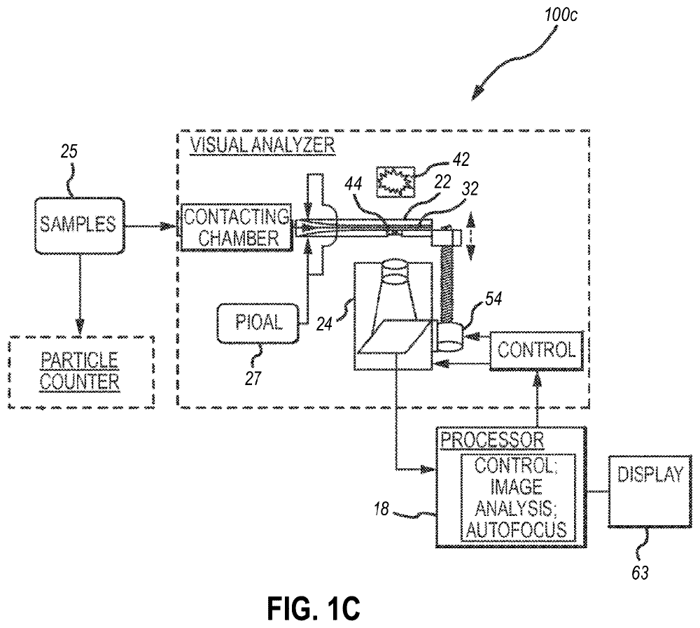

Referring now to FIG. 1, a schematical example of a flow cell 22 shown. In some embodiments, the flow cell 22 may convey a sample fluid through a viewing zone 23 of a high optical resolution imaging device 24 in a configuration for imaging microscopic particles in a sample flow stream 32 using digital image processing. Flow cell 22 may be coupled to a source 25 of sample fluid which may have been subjected to processing, such as contact with a particle contrast agent composition and heating. Flow cell 22 is also coupled to one or more sources of a particle and/or intracellular organelle alignment liquid (PIOAL) 27 also known as a sheath fluid, such as a clear glycerol solution having a viscosity that is greater than the viscosity of the sample fluid. In some embodiments, PIOAL includes iminodiac, a plurality of salts, bronidox, glycerol, and polyvinylpyrrolidone (PVP). Additional information on PIOAL/sheath fluid is provided in U.S. Pat. No. 9,316,635, entitled “Sheath fluid systems and methods for particle analysis in blood samples,” issued on Apr. 19, 2016, the disclosure of which is hereby incorporated by reference in its entirety.

The sample fluid is injected through a flattened opening at a distal end 28 of a sample feed tube 29, and into the interior of the flow cell 22 at a point where the PIOAL flow has been substantially established resulting in a stable and symmetric laminar flow of the PIOAL above and below (or on opposing sides of) the ribbon-shaped sample stream 32. The sample and PIOAL streams may be supplied by precision metering pumps that move the PIOAL with the injected sample fluid along a flowpath that narrows substantially. The PIOAL envelopes and compresses the sample fluid in the zone 21 where the flowpath narrows. Hence, the decrease in flowpath thickness at zone 21 can contribute to a geometric focusing of the sample stream 32. The sample fluid ribbon 32 is enveloped and carried along with the PIOAL downstream of the narrowing zone 21, passing in front of, or otherwise through the viewing zone 23 of, the high optical resolution imaging device 24 where images are collected, for example, using a CCD 48. In this way, flow imaging is performed where images from the flowing sample stream and the cellular material contained therein are collected. Processor 18 can receive, as input, pixel data from CCD 48. The sample fluid ribbon flows together with the PIOAL to a discharge 33.

As shown in FIG. 1, the narrowing zone 21 can have a proximal flowpath portion 21a having a proximal thickness PT and a distal flowpath portion 21b having a distal thickness (DT), such that distal thickness DT is less than proximal thickness (PT). The sample fluid can therefore be injected through the distal end 28 of sample tube 29 at a location that is distal to the proximal portion 21a and proximal to the distal portion 21b. Hence, the sample fluid can enter the PIOAL envelope as the PIOAL stream is compressed by the zone 21, wherein the sample fluid injection tube has a distal exit port through which sample fluid is injected into flowing sheath fluid, the distal exit port bounded by the decrease in flowpath size of the flow cell.

The digital high optical resolution imaging device 24 with objective lens 46 is directed along an optical axis that intersects the ribbon-shaped sample stream 32. The relative distance between the objective 46 and the flow cell 22 is variable by operation of a motor drive 54, for resolving and collecting a focused digitized image on a photosensor array. Additional information regarding the construction and operation of an exemplary flow cell such as shown in FIG. 1 is provided in U.S. Pat. No. 9,322,752, entitled “Flowcell Systems and Methods for Particle Analysis in Blood Samples,” issued on Apr. 26, 2016, the disclosure of which is hereby incorporated by reference in its entirety; and/or U.S. Pat. No. 9,857,361, entitled “Flowcell, Sheath Fluid, and Autofocus Systems and Methods for Particle Analysis in Urine Samples,” issued on Jan. 2, 2018, the disclosure of which is hereby incorporated by reference in its entirety. The embodiment of FIG. 1 represents a flow imaging system where cells are imaged under flow through flow cell 22.

Some embodiments may implement a technique for automatically achieving a correct working position of the high optical resolution imaging device 24 for focusing on the ribbon-shaped sample stream 32. The flowcell structure 22 can be configured such that the ribbon-shaped sample stream 32 has a fixed and dependable location within the flowcell defining the flow path of sample fluid, in a thin ribbon between layers of PIOAL, passing through a viewing zone 23 in the flowcell 22. In certain flowcell embodiments, the cross section of the flowpath for the PIOAL narrows symmetrically at the point at which the sample is inserted through a flattened orifice such as a tube 29 with a rectangular lumen at the orifice, or cannula. The narrowing flowpath (for example geometrically narrowing in cross sectional area by a ratio of 20:1, or by a ratio between 20:1 to 70:1) along with a differential viscosity between the PIOAL and sample fluids, and optionally, a difference in linear speed of the PIOAL compared to the flow of the sample, cooperate to compress the sample cross section by a ratio of about 20:1 to 70:1. In some embodiments the cross section thickness ratio may be 40:1.

In one aspect, the symmetrical nature of the flowcell 22 and the manner of injection of the sample fluid and PIOAL provide a repeatable position within the flowcell 22 for the ribbon-shaped sample stream 32 between the two layers of the PIOAL. As a result, process variations such as the specific linear velocities of the sample and the PIOAL; do not tend to displace the ribbon-shaped sample stream from its location in the flow. Relative to the structure of the flowcell 22, the ribbon-shaped sample stream 32 location is stable and repeatable.

However, the relative positions of the flowcell 22 and the high optical resolution imaging device 24 of the optical system may be subject to change and may benefit from occasional position adjustments to maintain an optimal or desired distance between the high optical resolution imaging device 24 and the ribbon-shaped sample stream 32, thus providing a quality focus image of the enveloped particles in the ribbon-shaped sample stream 32.

According to some embodiments, there can be an optimal or desired distance between the high optical resolution imaging device 24 and the ribbon-shaped sample stream 32 for obtaining focused images of the enveloped particles. The optics can first be positioned accurately relative to the flowcell 22 by autofocus or other techniques to locate the high optical resolution imaging device 24 at the optimal or desired distance from an autofocus target 44 with a fixed position relative to the flowcell 22. The displacement distance between the autofocus target 44 and the ribbon-shaped sample stream 32 is known precisely, for example as a result of initial calibration steps. After autofocusing on the autofocus target 44, the flowcell 22 and/or high optical resolution imaging device 24 is then displaced over the known displacement distance between the autofocus target 44 and the ribbon-shaped sample stream 32. As a result, the objective lens of the high optical resolution imaging device 24 is focused precisely on the ribbon-shaped sample stream 32 containing the enveloped particles.

Some embodiments may involve autofocusing on the focus or imaging target 44, which is a high contrast figure defining a known location along the optical axis of the high optical resolution imaging device or the digital image capture device 24. The target 44 can have a known displacement distance relative to the location of the ribbon-shaped sample stream 32. A contrast measurement algorithm can be employed specifically on the target features. In one example, the position of the high optical resolution imaging device 24 can be varied along a line parallel to the optical axis of the high optical resolution imaging device or the digital image capture device, to find the depth or distance at which one or more maximum differential amplitudes are found among the pixel luminance values occurring along a line of pixels in the image that is known to cross over an edge of the contrast figure. In some cases, the autofocus pattern has no variation along the line parallel to the optical axis, which is also the line along which a motorized control operates to adjust the position of the high optical resolution imaging device 24 to provide the recorded displacement distance.

In this way, it may not be necessary to autofocus or rely upon an image content aspect that is variable between different images, which is less highly defined as to contrast, or that might be located somewhere in a range of positions, as the basis for determining a distance location for reference. Having found the location of optimal or desired focus on the autofocus target 44, the relative positions of the high optical resolution imaging device objective 24 and the flowcell 22 can be displaced by the recorded displacement distance to provide the optimal or desired focus position for particles in the ribbon-shaped sample stream 32.

According to some embodiments, the high optical resolution imaging device 24 can resolve an image of the ribbon-shaped sample stream 32 as backlighted by a light source 42 applied through an illumination opening (window) 43. In the embodiments shown in FIG. 1, the perimeter of the illumination opening 43 forms an autofocusing target 44. However, the object is to collect a precisely focused image of the ribbon-shaped sample stream 32 through high optical resolution imaging device optics 46 on an array of photosensitive elements, such as an integrated charge coupled device.

The high optical resolution imaging device 24 and its optics 46 are configured to resolve an image of the particles in the ribbon-shaped sample stream 32 that is in focus at distance 50, which distance can be a result of the dimensions of the optical system, the shape of the lenses, and the refractive indices of their materials. In some cases, the optimal or desired distance between the high optical resolution imaging device 24 and the ribbon-shaped sample stream 32 does not change. In other cases, the distance between the flowcell 22 and the high optical resolution imaging device and its optics 46 can be changed. Moving the high optical resolution imaging device 24 and/or flowcell 22 closer or further apart, relative to one another (e.g., by adjusting distance 50 between the imaging device 24 and the flowcell 22), moves the location of the focusing point at the end of distance 50 relative to the flowcell.

In some embodiments, a focus target 44 can be located at a distance from the ribbon-shaped sample stream 32, in this case fixed directly to the flowcell 22 at the edges of the opening 43 for light from illumination source 42. The focus target 44 is at a constant displacement distance 52 from the ribbon-shaped sample stream 32. Often, the displacement distance 52 is constant because the location of the ribbon-shaped sample stream 32 in the flowcell remains constant.

An exemplary autofocus procedure involves adjusting the relative positions of the high optical resolution imaging device 24 and flowcell 22 using a motor 54 to arrive at the appropriate focal length thereby causing the high optical resolution imaging device 24 to focus on the autofocus target 44. By way of example, the relative position adjustment is done by moving one or more of the imaging device 24, the flowcell 22, or an objective of the imaging device so as to change the relative position between imaging device 24 and flowcell 22. In this example, the autofocus target 44 is behind the ribbon-shaped sample stream 32 in the flowcell. Then the high optical resolution imaging device 24 is moved toward or away from flowcell 22 until autofocus procedures establish that the image resolved on photosensor is an accurately focused image of autofocus target 44. Then motor 54 is operated to displace the relative positions of high optical resolution imaging device 24 and flowcell 22 to cause the high optical resolution imaging device to focus on the ribbon-shaped sample stream 32, namely by moving the high optical resolution imaging device 24 away from flowcell 22, precisely by the span of the displacement distance 52. In this exemplary embodiment, imaging device 24 is shown to be moved by motor 54 to get to a focus position. In another embodiments, an objective of imaging device 24 is moved. In other embodiments, flowcell 22 is moved or both the flowcell 22 and imaging device 24 are moved by similar means to obtain focused images.

These directions of movement would be reversed if the focus target 44 was located on the front viewport window as opposed to the rear illumination window 43. In that case, the displacement distance would be the span between the ribbon-shaped sample stream 32 and a target 44 at the front viewport (not shown).

The displacement distance 52, which is equal to the distance between ribbon-shaped sample stream 32 and autofocus target 44 along the optical axis of the high optical resolution imaging device 24, can be established in a factory calibration step or established by a user. Typically, once established, the displacement distance 52 does not change. Thermal expansion variations and vibrations may cause the precise position of the high optical resolution imaging device 24 and flowcell 22 to vary relative to one another, thus necessitating re-initiation of the autofocus process. But autofocusing on the target 44 provides a position reference that is fixed relative to the flowcell 22 and thus fixed relative to the ribbon-shaped sample stream 32. Likewise, the displacement distance is constant. Therefore, by autofocusing on the target 44 and displacing the high optical resolution imaging device 24 and flowcell 22 by the span of the displacement distance, the result is the high optical resolution imaging device being focused on the ribbon-shaped sample stream 32.

According to some embodiments, the focusing target 44 is provided as a high contrast circle printed or applied around the illumination opening 43. Alternative focusing target configurations are discussed elsewhere herein. When a square or rectangular image is collected in focus on the target 44, a high contrast border appears around the center of illumination. Seeking the position at which the highest contrast is obtained in the image at the inner edges of the opening, automatically focuses the high optical resolution imaging device 24 at the working location of the target 44. According to some embodiments, the term “working distance” can refer to the distance between the objective and its focal plane and the term “working location” can refer to the focal plane of the imaging device. The highest contrast measure of an image is where the brightest white and darkest black measured pixels are adjacent to one another along a line through an inner edge. The highest contrast measure can be used to evaluate whether the focal plane of the imaging device 24 is in the desired position relative to the target 44.

Other autofocus techniques can be used as well, such as edge detection techniques, image segmentation, and integrating the differences in amplitude between adjacent pixels and seeking the highest sum of differences. In one technique, the sum of differences is calculated at three distances that encompass working positions on either side of the target 44 and matching the resulting values to a characteristic curve, wherein the optimal distance is at the peak value on the curve. Relatedly, exemplary autofocus techniques can involve collecting images of the flow cell target at different positions and analyzing the images to find the best focus position using a metric that is largest when the image of the target is sharpest. During a first step (e.g., coarse step) the autofocus technique can operate to find a preliminary best position from a set of images collected at 2.5 μm intervals. From that position the autofocus technique can then involve collecting a second set of images (fine) at 0.5 μm intervals and calculating the final best focus position on the target.

In some cases, the focus target 44 (e.g., autofocus pattern) can reside on the periphery of the area of view in which the sample is to appear. It is also possible that the focus target 44 could be defined by contrasting shapes that reside in the field of view. Typically, the autofocus target 44 is mounted on the flowcell 22 or attached rigidly in fixed position relative to the flowcell. Under power of a positioning motor 54 controlled by a detector (e.g., processor 18) responsive to maximizing the contrast of the image of the autofocusing target, the apparatus autofocuses on the target 44 as opposed to the ribbon-shaped sample stream. Then by displacing the flowcell 22 and/or the high optical resolution imaging device 24 relative to one another, by the displacement distance known to be the distance between the autofocus target 44 and the ribbon-shaped sample stream 32, the working position or the focal plane of the high optical resolution imaging device is displaced from the autofocus target to the ribbon-shaped sample stream. As a result, the ribbon-shaped sample stream 32 appears in focus in the collected digital image.

In some embodiments, an additional focusing step is used after the target autofocus step. For instance, the focusing to a target is a first step to establish a general position of a position of a camera relative to a flowcell/target of a flowcell. An additional step can utilize real-time focusing to imaged samples (e.g., blood cells). One example includes a pixel binning analysis among V/brightness-values of red blood cells or white blood cells, and a comparison of V-values between the various bins to establish an ideal focal location. Alternatively, after the target is used to set a location of the camera relative to the flowcell/target of the flowcell, a focal assessment step to gauge focal quality of images post-acquisition can occur to monitor camera focal position over time-such as utilizing the V/brightness-values or red or white blood cells as described herein. Further information about automatic focusing approaches which may be implemented in some embodiments is provided in U.S. Pat. Nos. 9,857,361, 10,705,008, 10,705,011, international patent application PCT/US2022/052702, and international patent application PCT/US2023/011759, the contents of each of which are hereby incorporated by reference in their entirety.

In order to distinguish particle types by data processing techniques, such as categories and/or subcategories of red and white blood cells, it is advantageous to record microscopic pixel images that have sufficient resolution and clarity to reveal the aspects that distinguish one category or subcategory from the others.

In an embodiment, the apparatus can be based on an optical bench arrangement such as shown in FIG. 1A and as enlarged in FIG. 1B, having a source of illumination 42 directed onto a flowcell 22 mounted in a gimbaled or flowcell carrier 55, backlighting the contents of the flowcell 22 in an image obtained by a high optical resolution imaging device 24. Carrier 55 is mounted on a motor drive so as to be precisely movable toward and away from the high optical resolution imaging device 24. Carrier 55 also allows a precise alignment of the flowcell 22 relative to the optical viewing axis of the high optical resolution imaging device or the digital image capture device 24, so that the ribbon-shaped sample stream flows in a plane normal to the viewing axis in the zone where the ribbon-shaped sample stream is imaged, namely between the illumination opening 43 and viewing port 57 as depicted in FIG. 1. The focus target 44 can assist in adjustment of carrier 55, for example to establish the plane of the ribbon-shaped sample stream normal to the optical axis of the high optical resolution imaging device or the digital image capture device.

Accordingly, carrier 55 may provide for very precise linear and angular adjustment of the position and orientation of flowcell 22, for example relative to the image capture device 24 or the image capture device objective. As shown here, the carrier 55 may include two pivot points 55a and 55b to facilitate angular adjustment of the carrier and flowcell 22 relative to the image capture device 24. Angular adjustment pivot points 55a and 55b may be located in the same plane and centered to the flow cell 22 channel (e.g., at the image capture site). This allows for adjustment of the angles without causing any linear translation of the flow cell 22 position. The carrier 55 can be rotated about an axis of pivot point 55a or about an axis of pivot point 55b, or about both axes. Such rotation can be controlled by a processor 18 and a flowcell movement control mechanism (e.g., motor 54).

With continued reference to FIG. 1B, it can be seen that either or both of the image capture device 24 and/or the carrier 55 (along with flowcell 22) can be rotated or translated along various axes (e.g., X, Y, Z) in three dimensions. Thus, in some embodiments, a technique for adjusting focus of the image capture device may include implementing axial rotation of the image capture device 24 about the imaging axis, for example by rotating device about axis X. In a further embodiment, focus adjustment can also be achieved by axial rotation of the flowcell 22 and/or carrier 55 about an axis extending along the imaging axis, for example about axis X, and within the field of view of the imaging device 24.

In some cases, focus adjustment may include tip rotation (e.g., rotation about axis Y) of the image capture device. In other cases, the focus adjustment may include tip rotation (e.g., rotation about axis Y, or about pivot point 55a) of the flowcell 22. As depicted here, pivot point 55a corresponds to a Y axis that extends along and within the flowpath of the flowcell. In some cases, focus adjustment can include tilt rotation (e.g., rotation about axis Z) of the image capture device. In other cases, the focus adjustment may include tilt rotation (e.g., rotation about axis Z, or about pivot point 55b) of the flowcell 22. As shown in FIG. 1B, the pivot point 55b corresponds to a Z axis that traverses the flowpath and the imaging axis. In some cases, the image capture device 24 can be focused on the sample flowstream by implementing a rotation of the flowcell 22 (e.g., about axis X), such that the rotation is centered in the field of view of the image capture device. The three-dimensional rotational adjustments described herein can be implemented so as to account for positional drift in one or more components of the analyzer system. In some embodiments, the three-dimensional rotational adjustments can be implemented so as to account for temperature fluctuations in one or more components of the analyzer system. In additional embodiments, the adjustment of an analyzer system may include translating imaging device 24 along axis X. Additionally, in some embodiments, the adjustment of analyzer system may include translating carrier 55 or flowcell 22 along axis X. Further information on such a carrier which may be utilized in some embodiments is provided in U.S. patent application Ser. No. 18/224,953, the disclosure of which is hereby incorporated by reference in its entirety.

Thus, according to the one or more embodiments disclosed herein, a visual analyzer for obtaining images of a sample containing particles suspended in a liquid includes flowcell 22, coupled to a source 25 of the sample and to a source 27 of PIOAL material as depicted in FIG. 1. The flowcell 22 may define an internal flowpath that narrows symmetrically in the flow direction. The flowcell 22 is configured to direct a flow 32 of the sample enveloped with the PIOAL through a viewing zone in the flowcell, namely behind viewing port 57. Furthermore, referring again to FIG. 1, the digital high optical resolution imaging device 24 with objective lens 46 may be directed along an optical axis that intersects the ribbon-shaped sample stream 32. The relative distance between the objective 46 and the flowcell 22 may be variable by operation of a motor drive 54, for resolving and collecting a focused digitized image on a photosensor array.

The autofocus target 44, having a position that is fixed relative to the flowcell 22, is located at a displacement distance 52 from the plane of the ribbon-shaped sample stream 32. In the embodiment shown, the autofocus target 44 is applied directly to the flowcell 22 at a location that is visible in the image collected by the high optical resolution imaging device 24. In another embodiment, the autofocus target may be carried on a part that is rigidly fixed in position relative to the flowcell 22 and the ribbon-shaped sample stream 32 therein, if not applied directly to the body of the flowcell in an integral manner.

The light source 42, which can be a steady source or can be a strobe that is flashed in time with operation of the high optical resolution imaging device photosensor, is configured to illuminate the ribbon-shaped sample stream 32 and also to contribute to the contrast of the target 44. In the depicted embodiment, the illumination is from back-lighting. In some examples, the light source 42 can include a single light (e.g., LED) or a plurality of lights (e.g. 3 LED's-one green, one red, one blue which are combined to create a single white light). Further information on how lighting may be provided in some implementations is provided in U.S. patent application Ser. No. 18/224,937, the disclosure of which is hereby incorporated by reference in its entirety.

Referring now to FIG. 1C, a block diagram of additional aspects of a hematology analyzer 100c is shown. In some embodiments, and as shown, the analyzer 100c may include at least one digital processor 18 coupled to operate the motor drive 54 and to analyze the digitized image from the photosensor array as collected at different focus positions relative to the target autofocus pattern 44. The processor 18 is configured to determine a focus position of the autofocus pattern 44 (e.g., to autofocus on the target autofocus pattern 44 and thus establish an optimal distance between the high optical resolution imaging device 24 and the autofocus pattern 44). In some embodiments, this may be accomplished by image processing steps such as applying an algorithm to assess the level of contrast in the image at a first distance, which can apply to the entire image or at least at an edge of the autofocus pattern 44. The processor moves the motor 54 to another position and assesses the contrast at that position or edge, and after two or more iterations determines an optimal distance that maximizes the accuracy of focus on the autofocus pattern 44 (or would optimize the accuracy of focus if moved to that position). The processor may rely on the fixed spacing between the autofocus target 44 and the ribbon-shaped sample stream 32, the processor 18 may then control the motor 54 to move the high optical resolution imaging device 24 to the correct distance to focus on the ribbon-shaped sample stream 32. More particularly, the processor 18 may operates the motor 54 to displace the distance 50 between the high optical resolution imaging device 24 and the ribbon-shaped sample stream 32 by the displacement distance 52 (for example as depicted in FIG. 1) by which the ribbon-shaped sample stream is displaced from the target autofocus pattern 44. In this way, the high optical resolution imaging device is focused on the ribbon-shaped sample stream.

The flowcell internal contour and the PIOAL and sample flow rates can be adjusted such that the sample is formed into a ribbon shaped stream 32. The stream can be approximately as thin as or even thinner than the particles that are enveloped in the ribbon-shaped sample stream. White blood cells may have a diameter around 10 μm, for example. By providing a ribbon-shaped sample stream 32 with a thickness less than 10 μm, the cells may be oriented when the ribbon-shaped sample stream is stretched by the sheath fluid, or PIOAL. Surprisingly stretching of the ribbon-shaped sample stream along a narrowing flowpath within PIOAL layers of different viscosity than the ribbon-shaped sample stream, such as higher viscosity, advantageously tends to align non-spherical particles in a plane substantially parallel to the flow direction, and apply forces on the cells, improving the in-focus contents of intracellular structures of cells. The optical axis of the high optical resolution imaging device 24 is substantially normal (i.e., perpendicular) to the plane of the ribbon-shaped sample stream 32. The linear velocity of the ribbon-shaped sample stream 32 at the point of imaging may be, for example, 20-200 mm/second. In some embodiments, the linear velocity of the ribbon-shaped sample stream may be, for example, 50-150 mm/second.

The ribbon-shaped sample stream thickness can be affected by the relative viscosities and flow rates of the sample fluid and the PIOAL. With returning reference to FIG. 1, the source 25 of the sample and/or the source 27 of the PIOAL, for example comprising precision displacement pumps and/or optimized flow restrictor tubing dimensions along with a single fluid source for driving relevant fluid flow, can be configured to provide the sample and/or the PIOAL at controllable and optimized flow rates for optimizing the dimensions of the ribbon-shaped sample stream 32, namely as a thin ribbon at least as wide as the field of view of the high optical resolution imaging device 24. Further information on approaches to sample driving which may be utilized in some embodiments is provided in international patent application PCT/US2022/054240, the disclosure of which is hereby incorporated by reference in its entirety. In one example, PIOAL is contained in a single tank which has two flowpaths—a first flowpath delivers the PIOAL to the flowcell, the second flowpath delivers the PIOAL in the vicinity of a specimen sample entry point near the flowcell where the PIOAL is then used to push the specimen sample through the flowcell. Flow restrictors are configured on each flowpath to affect the relative speed/flow in each flowpath, and the use of a single PIOAL source ensures that a speed/flow ratio between the sample and PIOAL flow is relatively constant.

In one embodiment, the source 27 of the PIOAL is configured to provide the PIOAL at a predetermined viscosity. That viscosity may be different than the viscosity of the sample and can be higher than the viscosity of the sample. The viscosity and density of the PIOAL, the viscosity of the sample material, the flow rate of the PIOAL and the flow rate of the sample material are coordinated to maintain the ribbon-shaped sample stream at the displacement distance from the autofocus pattern, and with predetermined dimensional characteristics, such as an advantageous ribbon-shaped sample stream thickness. In a further embodiment, the PIOAL may have a higher linear velocity than the sample and a higher viscosity than the sample, thereby stretching the sample into the flat ribbon. In some cases, the PIOAL viscosity can be up to 10 centipoise.

In the embodiment shown in FIG. 1C, the same digital processor 18 that is used to analyze the pixel digital image obtained from photosensor array may also be used to control the autofocusing motor 54. However, typically the high optical resolution imaging device 24 is not autofocused for every image captured. The autofocus process can be accomplished periodically (at the beginning of the day or at the beginning of a shift) or for example when temperature or other process changes are detected by appropriate sensors, or when image analysis detects a potential need for refocusing. In some cases, an automated autofocusing process may be performed within a time duration of about 10 seconds. In some cases, an autofocus procedure can be performed prior to processing a rack of samples (e.g., 10 samples per rack). It is also possible in other embodiments to have the hematology image analysis accomplished by one processor and to have a separate processor, optionally associated with its own photosensor array, arranged to handle the steps of autofocusing to a fixed target 44.

The digital processor 18 can be configured to autofocus at programmed times or in programmed conditions or on user demand, and also is configured to perform image-based categorization and subcategorization of the particles. Exemplary particles include cells, white blood cells, red blood cells and the like. In one embodiment, the digital processor 18 is configured to detect an autofocus re-initiation signal. The autofocus re-initiation signal can be triggered by a detected change in temperature, a decrease in focus quality as discerned by parameters of the pixel image date, passage of time, or user-input. Advantageously, it is not necessary to recalibrate in the sense of measuring the displacement distance 52 depicted in FIG. 1 to recalibrate. Optionally, the autofocus can be programmed to re-calibrate at certain frequencies/intervals between runs for quality control and or to maintain focus.

The displacement distance 52 varies slightly from one flowcell to another but remains constant for a given flowcell. As a setup process when fitting out an image analyzer with a flowcell, the displacement distance is first estimated and then during calibration steps wherein the autofocus and imaging aspects are exercised, the exact displacement distance for the flowcell is determined and entered as a constant into the programming of processor 18. In further embodiments, the processor 18 may present on a display 63 various information for the user to review and/or analyze, as will be discussed further herein.

As mentioned above, some systems may include an imaging system/module having a flow cell 22, a high optical resolution imaging device 24, and a processor 18, which, in conjunction with each other and other suitable components, are configured to utilize a sample fluid (e.g., a patient sample) in order to cooperatively (A) collect quality images of microscopic particles in a sample flow stream 32 using digital image processing, (B) record such collected images, and (C) process collected digital images utilizing suitable data processing techniques as would be apparent to one skilled in the art in view of the teachings herein (e.g., categorize such microscopic particles into various suitable categories and/or subcategories). In other words, imaging systems/modules similar to those described above may be utilized to obtain information about a sample fluid via high quality images of microscopic particles within the sample fluid. For example, static or slide-based imaging can be used instead of the flow-imaging and flowcell-imaging based concepts described above and herein.

Imaging Systems Combined with Alternative Systems

In addition to the imaging based systems and modules described herein, some systems/modules may obtain information from a sample fluid via means other than capturing high quality images of microscopic particles in a sample flow stream 32. Such systems/modules may utilize, for example, impedance systems, fluorescence systems, light scatter systems, VCS systems (integration of volume, conductivity, and scatter together), spectrophotometry systems, or any other suitable systems as would be apparent to one skilled in the art in view of the teachings herein. Such systems may be referred to as alternative systems or “non-imaging”, as those systems may not capture high quality images of microscopic particles. Some alternative systems may include systems that utilize a different imaging analysis process (e.g., different than the flow imaging described herein) to obtain data, etc. Alternative systems may collect sample fluid information including identical, similar, and/or different parameters compared to the information obtained by imaging systems described above.

These alternative systems may be helpful in order to obtain certain particle information that may be difficult to derive from images. For example, the imaging system may not be able to assess volumetric data related to cells, and thus an alternative system may need to be included with the imaging system in order to establish this volumetric data. In another example, the imaging system may not be able to assess hemoglobin content from images and therefore a separate hemoglobin module (e.g., spectrophotometer) is included as an additional module. These alternative systems can also be used to provide a second set of parameters for result verification (e.g., counting red blood cells with an imaging based analytical system, and a non-imaging based analytical system).

In some embodiments, an analyzer or analysis system would utilize multiple channels—a first imaging channel (e.g., flow imaging), and one or more non-imaging channel (e.g., one or more of impedance, fluorescence, spectrophotometry, conductivity, light scatter, or volume-conductivity-scatter (VCS)). Each channel can also be considered as a module, such that there is an imaging module, and one or more non-imaging modules. In one example, an analyzer or analysis system utilizes a flow imaging channel/module, an impedance channel/module, and spectrophotometry channel/module.

In some embodiments, a second non-imaging channel can utilize a plurality of non-imaging modules therein (e.g., combinations of impedance, conductivity, light scatter, VCS, fluorescence, and spectrophotometry). In other words, there is a dedicated imaging channel, and a dedicated non-imaging channel where all the non-imaging analysis is done on the particular channel. In one example, an analyzer or analysis system utilizes two channels—a first flow imaging channel, and a second non-imaging channel utilizing a plurality of non-imaging modules including, for instance, an impedance and a spectrophotometry module. Additional explanation of these alternative or non-imaging modules, channels, or systems is provided herein.

### Impedance System

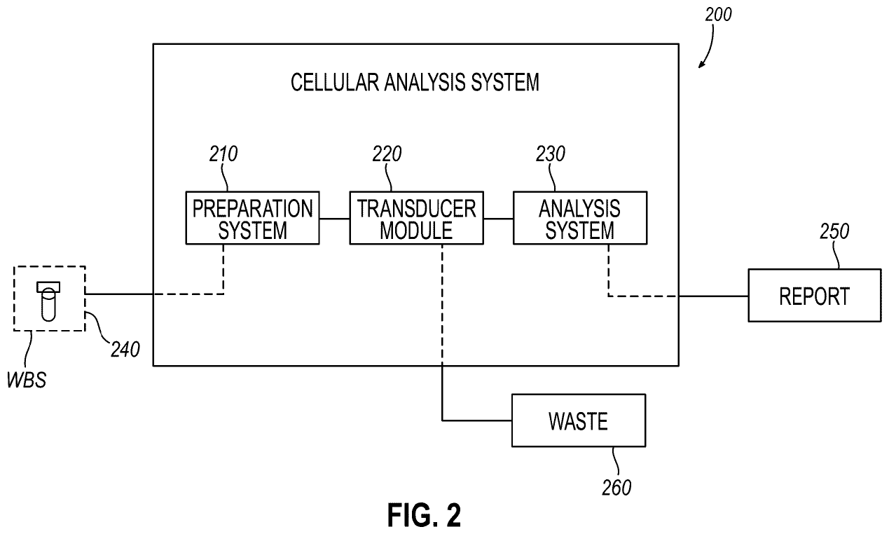

Referring now to FIG. 2, a schematic representation of a cellular analysis system 200 is shown. In some embodiments, and as shown, system 200 may include a preparation system 210, a transducer module 220, and an analysis system 230. While the system 200 is described herein at a very high level, with reference to the three core system blocks (e.g., 210, 220, and 230), the skilled artisan would readily understand that system 200 includes many other system components (such as discussed above with reference to FIGS. 1, 1B, and 1C) such as central control processor(s), display system(s), fluidic system(s), temperature control system(s), user-safety control system(s), and the like. In operation, a fluid sample (e.g., a whole blood sample (WBS)) 240 can be presented to the system 200 for analysis. In some instances, the sample 240 is aspirated into system 200. Exemplary aspiration techniques are known to the skilled artisan. After aspiration, the sample 240 can be delivered to a preparation system 210. Preparation system 210 receives the sample 240 and can perform operations involved with preparing the sample 240 for further measurement and analysis. For example, preparation system 210 may separate the sample 240 into predefined aliquots for presentation to transducer module 220. Preparation system 210 may also include mixing chambers so that appropriate reagents may be added to the aliquots. For example, where an aliquot is to be tested for differentiation of white blood cell subset populations, a lysing reagent (e.g., ERYTHROLYSE, a red blood cell lysing buffer) may be added to the aliquot to break up and remove the Red Blood Cells (RBCs). Preparation system 210 may also include temperature control components (not shown) to control the temperature of the reagents and/or mixing chambers. Appropriate temperature controls can improve the consistency of the operations of preparation system 210. As discussed elsewhere herein, sample data such as light scatter data, light absorption data, and/or current data can be obtained (e.g., using a transducer) and processed or used to determine various blood cell status indications of an individual patient.

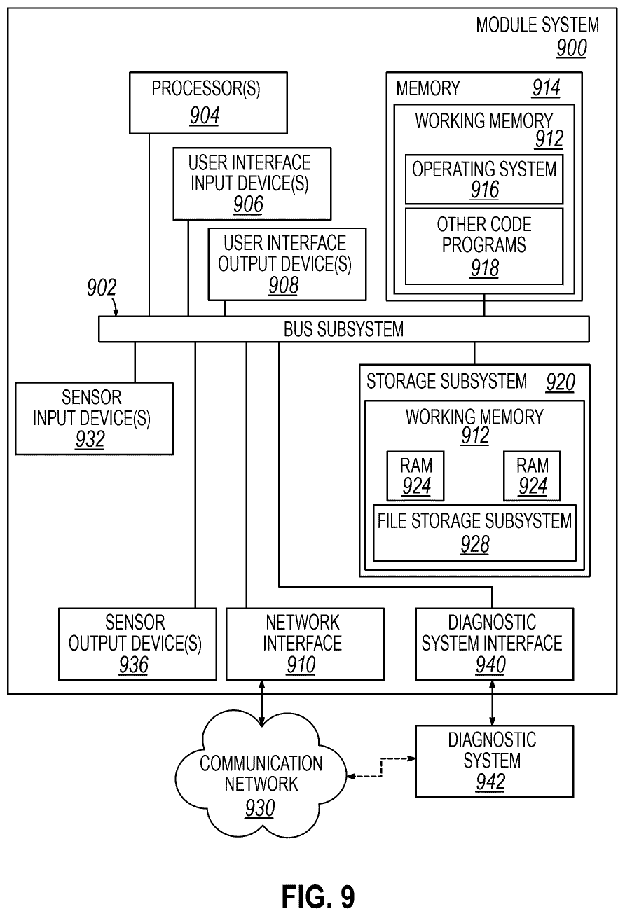

In some instances, predefined aliquots can be transferred from preparation system 210 to transducer module 220. As described in further detail below, transducer module 220 may be able to perform direct current (DC) impedance, radiofrequency (RF) conductivity, light transmission, and/or light scatter measurements of cells from the sample 240 passing individually therethrough. Measured DC impedance, RF conductivity, and light propagation (e.g., light transmission, light scatter) parameters can be provided or transmitted to analysis system 230 for data processing. In some instances, analysis system 230 may include computer processing features and/or one or more modules or components such as those described herein with reference to the system depicted in FIG. 9 and described further below, which can evaluate the measured parameters, identify and enumerate the blood cellular constituents, and correlate a subset of data characterizing elements of the sample 240 with a White Blood Cell Count (WBC) status of the individual. As shown here, cellular analysis system 200 may generate or output a report 250 containing the predicted status and/or a prescribed treatment regimen for the individual. In some instances, excess biological sample from transducer module 220 can be directed to an external (or alternatively internal) waste system 260.

In one embodiment transducer module 220 comprises an impedance detector which utilizes impedance, also known as the Coulter principle, to count individual cells as they pass through an aperture (correlating a displacement, and corresponding electrical response to cell size/volume). In one embodiment, the impedance detector is configured to measure one or more of red blood cells, white blood cells, and platelets. In one embodiment, the impedance detector is configured to measure red blood cells and platelets (e.g., configuring a threshold to only count cells in the range of a blood cell and platelet), mean corpuscular volume (average volume of red blood cells), and mean platelet volume (average volume of platelets).

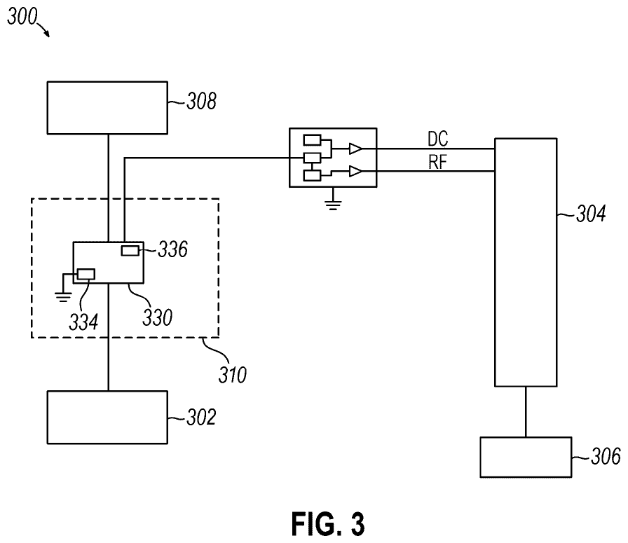

In the context of FIG. 3, which illustrates a transducer module in more detail (and references an impedance portion of a transducer module), there are electrodes 334, 336 for performing DC impedance measurements of cells passing through an interrogation zone (e.g., two tanks separated by an aperture which cells pass through). Signals from electrodes 334, 336 are transmitted to an analysis system 304 to process the data and establish a cell count and other numeric cell parameters (e.g., volumetric data). This data is then output to report 306. Any remaining fluid is discharged to waste 308.

In one example, the use of solely an impedance detector may have particular utility for red blood cells and platelets, or also counting white blood cells where discrimination between the various types of white blood cells is not needed. This is since it may be difficult to distinguish between various types of white blood cells (e.g., at least neutrophils, lymphocytes, monocytes, eosinophils, basophils) solely through an impedance measurement which would count the white blood cell and assess its size, but would need additional analysis to differentiate the type of white blood cell. By way of example, the impedance detector can be used on one or more of: red blood cell count, platelet count, mean corpuscular volume, mean platelet volume, and/or white blood cell count.

### Conductivity Systems

FIG. 3 illustrates in more detail a transducer module and associated components in more detail which includes a conductivity measurement. Note, FIG. 3 shows how impedance (DC) measurement and conductivity could be integrated within a single system. In some embodiments, and as shown, system 300 may include a transducer module 310 having a flow cell 330, which may include an electrode assembly having first and second electrodes 334, 336 for performing DC impedance and RF conductivity measurements of the cells passing through cell interrogation zone 332. Signals from electrodes 334, 336 can be transmitted to analysis system 304. The electrode assembly can analyze volume and conductivity characteristics of the cells using low-frequency current and high-frequency current, respectively. For example, low-frequency DC impedance measurements can be used to analyze the volume of each individual cell passing through the cell interrogation zone. Relatedly, high-frequency RF current measurements can be used to determine the conductivity of cells passing through the cell interrogation zone. Because cell walls act as conductors to high frequency current, the high frequency current can be used to detect differences in the insulating properties of the cell components, as the current passes through the cell walls and through each cell interior. High frequency current can be used to characterize nuclear and granular constituents and the chemical composition of the cell interior.

Wires or other transmission or connectivity mechanisms can transmit signals from the electrode assembly (e.g., electrodes 334, 336) to analysis system 304 for processing. For example, measured DC impedance or RF conductivity parameters can be provided or transmitted to analysis system 304 for data processing. In some instances, analysis system 304 may include computer processing features and/or one or more modules or components such as those described herein with reference to the system depicted in FIG. 9, which can evaluate the measured parameters, identify and enumerate biological sample constituents, and correlate a subset of data characterizing elements of the biological sample with a status of the individual. As shown here, cellular analysis system 300 may generate or output a report 306 containing the predicted status and/or a prescribed treatment regimen for the individual. In some instances, excess biological sample from transducer module 310 can be directed to an external (or alternatively internal) waste system 308. In some instances, a cellular analysis system 300 may include one or more features of a transducer module or blood analysis instrument such as those described in previously incorporated U.S. Pat. Nos. 5,125,737; 6,228,652; 8,094,299; and 8,189,187.

In some embodiments, a conductivity system can be standalone (e.g., would not include an impedance detector), or could be paired with an impedance detector to provide additional particle information.

### Light Scatter Systems

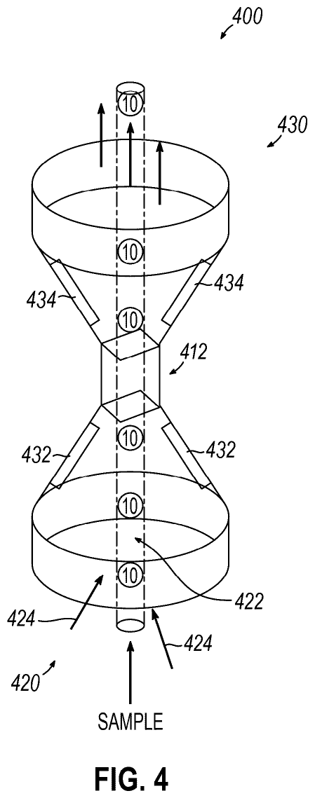

FIG. 4 illustrates aspects of an automated cellular analysis system for predicting or assessing a type of white blood cell (WBC). In particular, the WBC can be assessed based on a biological sample obtained from blood of the individual. As shown here, an analysis system or transducer 400 may include an optical element 410 having a cell interrogation zone 412. The transducer also provides a flow path 420, which delivers a hydrodynamically focused stream 422 of a biological sample toward the cell interrogation zone 412. For example, as the sample stream 422 is projected toward the cell interrogation zone 412, a volume of sheath fluid 424 can also enter the optical element 410 under pressure, so as to uniformly surround the sample stream 422 and cause the sample stream 422 to flow through the center of the cell interrogation zone 412, thus achieving hydrodynamic focusing of the sample stream. In this way, individual cells of the biological sample, passing through the cell interrogation zone one cell at a time, can be precisely analyzed.

Note, for the purposes of illustration in the context of FIG. 4, light scatter analysis has been combined with direct current (DC) impedance and radiofrequency (RF) conductivity in a single module or system 400. Transducer module or system 400 also includes an electrode assembly 430 that measures direct current (DC) impedance and radiofrequency (RF) conductivity of cells 10 of the biological sample passing individually through the cell interrogation zone 412. The electrode assembly 430 may include a first electrode mechanism 432 and a second electrode mechanism 434. As discussed elsewhere herein, low-frequency DC measurements can be used to analyze the volume of each individual cell passing through the cell interrogation zone. Relatedly, high-frequency RF current measurements can be used to determine the conductivity of cells passing through the cell interrogation zone. Such conductivity measurements can provide information regarding the internal cellular content of the cells. For example, high frequency RF current can be used to analyze nuclear and granular constituents, as well as the chemical composition of the cell interior, of individual cells passing through the cell interrogation zone. Thus, in some embodiments, the DC and RF measurements can be made on cells passing through the cell interrogation zone. As described earlier, for the purposes of illustration the light scatter has been combined with DC and RF measurement in a single module or system 400. This may be desirable in some contexts to provide additional cell information (e.g., all the non-imaging based data) in one simplified structure. Alternative embodiments can have the light scatter by itself (i.e., not including impedance or conductivity), or can include combinations of impedance and conductivity as separate modules added on to a light scatter module. The principles of light scatter detection will now be explained further.

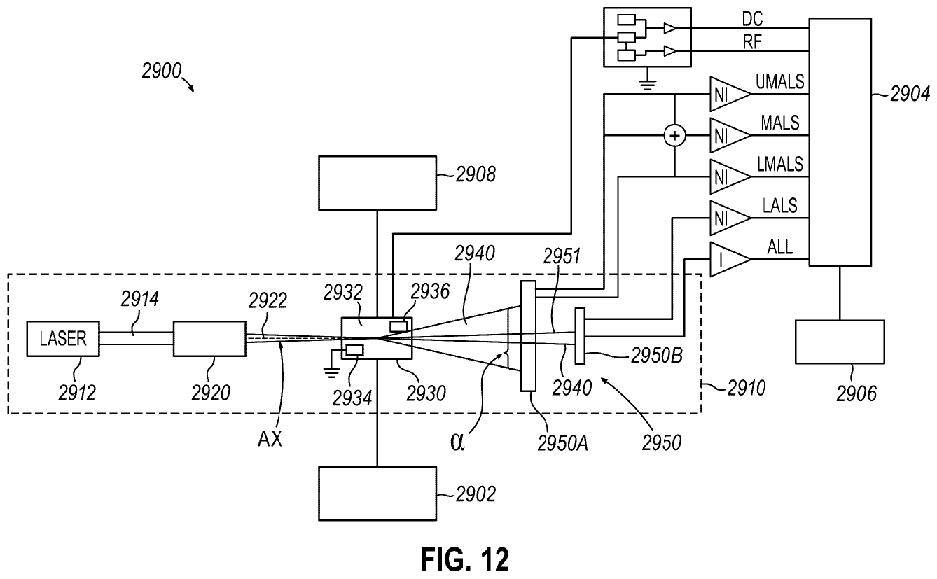

Turning now to FIG. 12, as illustrated in that figure, a cellular analysis system may include a transducer module 2910 having a light or irradiation source such as a laser 2910 emitting a beam 2914. The laser 2912 can be, for example, a 635 nm, 5 mW, solid-state laser. In some instances, system 2900 may include a focus-alignment system 2920 that adjusts beam 2914 such that a resulting beam 2922 is focused and positioned at a cell interrogation zone 2932 of a flowcell 2930. In some instances, flowcell 2930 receives a sample aliquot from a preparation system 2902. Note, as described earlier, the light scatter detection system is also illustratively shown with DC (impedance) and RF (conductivity), but can be a standalone system or module.

In some instances, the aliquot generally flows through the cell interrogation zone 2932 such that its constituents pass through the cell interrogation zone 2932 one at a time. In some cases, a system 2900 may include a cell interrogation zone or other feature of a transducer module or blood analysis instrument such as those described in U.S. Pat. Nos. 5,125,737; 6,228,652; 7,390,662; 8,094,299; and 8,189,187, the contents of each of which are incorporated herein by reference in their entirety. For example, a cell interrogation zone 2932 may be defined by a square transverse cross-section measuring approximately 50×50 microns, and having a length (measured in the direction of flow) of approximately 65 microns. Flow cell 2930 may include an electrode assembly having first and second electrodes 2934, 2936 for performing DC impedance and RF conductivity measurements of the cells passing through cell interrogation zone 2932. Signals from electrodes 2934, 2936 can be transmitted to analysis system 2904. The electrode assembly can analyze volume and conductivity characteristics of the cells using low-frequency current and high-frequency current, respectively. For example, low-frequency DC impedance measurements can be used to analyze the volume of each individual cell passing through the cell interrogation zone. Relatedly, high-frequency RF current measurements can be used to determine the conductivity of cells passing through the cell interrogation zone. Because cell walls act as conductors to high frequency current, the high frequency current can be used to detect differences in the insulating properties of the cell components, as the current passes through the cell walls and through each cell interior. High frequency current can be used to characterize nuclear and granular constituents and the chemical composition of the cell interior.

Incoming beam 2922 travels along beam axis AX and irradiates the cells passing through cell interrogation zone 2932, resulting in light propagation within an angular range a (e.g. scatter, transmission) emanating from the zone 2932. Exemplary systems are equipped with sensor assemblies that can detect light within three, four, five, or more angular ranges within the angular range a, including light associated with an extinction or axial light loss measure as described elsewhere herein. As shown here, light propagation 2940 can be detected by a light detection assembly 2950, optionally having a light scatter detector unit 2950A and a light scatter and transmission detector unit 2950B. In some instances, light scatter detector unit 2950A includes a photoactive region or sensor zone for detecting and measuring upper median angle light scatter (UMALS), for example light that is scattered or otherwise propagated at angles relative to a light beam axis within a range from about 20 to about 42 degrees. In some instances, UMALS corresponds to light propagated within an angular range from between about 20 to about 43 degrees, relative to the incoming beam axis which irradiates cells flowing through the interrogation zone. Light scatter detector unit 2950A may also include a photoactive region or sensor zone for detecting and measuring lower median angle light scatter (LMALS), for example light that is scattered or otherwise propagated at angles relative to a light beam axis within a range from about 10 to about 20 degrees. In some instances, LMALS corresponds to light propagated within an angular range from between about 9 to about 19 degrees, relative to the incoming beam axis which irradiates cells flowing through the interrogation zone.

A combination of UMALS and LMALS is defined as median angle light scatter (MALS), which is light scatter or propagation at angles between about 9 degrees and about 43 degrees relative to the incoming beam axis which irradiates cells flowing through the interrogation zone.

As shown in FIG. 12, the light scatter detector unit 2950A may include an opening 2951 that allows low angle light scatter or propagation 2940 to pass beyond light scatter detector unit 2950A and thereby reach and be detected by light scatter and transmission detector unit 2950B. According to some embodiments, light scatter and transmission detector unit 2950B may include a photoactive region or sensor zone for detecting and measuring lower angle light scatter (LALS), for example light that is scattered or propagated at angles relative to an irradiating light beam axis of about 5.1 degrees. In some instances, LALS corresponds to light propagated at an angle of less than about 9 degrees, relative to the incoming beam axis which irradiates cells flowing through the interrogation zone. In some instances, LALS corresponds to light propagated at an angle of less than about 10 degrees, relative to the incoming beam axis which irradiates cells flowing through the interrogation zone. In some instances, LALS corresponds to light propagated at an angle of about 1.9 degrees±0.5 degrees, relative to the incoming beam axis which irradiates cells flowing through the interrogation zone. In some instances, LALS corresponds to light propagated at an angle of about 3.0 degrees±0.5 degrees, relative to the incoming beam axis which irradiates cells flowing through the interrogation zone. In some instances, LALS corresponds to light propagated at an angle of about 3.7 degrees±0.5 degrees, relative to the incoming beam axis which irradiates cells flowing through the interrogation zone. In some instances, LALS corresponds to light propagated at an angle of about 5.1 degrees±0.5 degrees, relative to the incoming beam axis which irradiates cells flowing through the interrogation zone. In some instances, LALS corresponds to light propagated at an angle of about 7.0 degrees±0.5 degrees, relative to the incoming beam axis which irradiates cells flowing through the interrogation zone.

According to some embodiments, light scatter and transmission detector unit 2950B may include a photoactive region or sensor zone for detecting and measuring light transmitted axially through the cells, or propagated from the irradiated cells, at an angle of 0 degrees relative to the incoming light beam axis. In some cases, the photoactive region or sensor zone may detect and measure light propagated axially from cells at angles of less than about 1 degree relative to the incoming light beam axis. In some cases, the photoactive region or sensor zone may detect and measure light propagated axially from cells at angles of less than about 0.5 degrees relative to the incoming light beam axis less. Such axially transmitted or propagated light measurements correspond to axial light loss (ALL or AL2). As noted in previously incorporated U.S. Pat. No. 7,390,662, when light interacts with a particle, some of the incident light changes direction through the scattering process (i.e. light scatter) and part of the light is absorbed by the particles. Both of these processes remove energy from the incident beam. When viewed along the incident axis of the beam, the light loss can be referred to as forward extinction or axial light loss. Additional aspects of axial light loss measurement techniques are described in U.S. Pat. No. 7,390,662 at column 5, line 58 to column 6, line 4.

As such, the cellular analysis system 2900 provides means for obtaining light propagation measurements, including light scatter and/or light transmission, for light emanating from the irradiated cells of the biological sample at any of a variety of angles or within any of a variety of angular ranges, including ALL and multiple distinct light scatter or propagation angles. For example, light detection assembly 2950, including appropriate circuitry and/or processing units, provides a means for detecting and measuring UMALS, LMALS, LALS, MALS, and ALL.

Wires or other transmission or connectivity mechanisms can transmit signals from the electrode assembly (e.g. electrodes 2934, 2936), light scatter detector unit 2950A, and/or light scatter and transmission detector unit 2950B to analysis system 2904 for processing. For example, measured DC impedance, RF conductivity, light transmission, and/or light scatter parameters can be provided or transmitted to analysis system 2904 for data processing. In some instances, analysis system 2904 may include computer processing features and/or one or more modules or components such as those described herein, which can evaluate the measured parameters, identify and enumerate biological sample constituents, and correlate a subset of data characterizing elements of the biological sample with an infection status of the individual. As shown here, cellular analysis system 2900 may generate or output a report 2906 containing the evaluated infection status and/or a prescribed treatment regimen for the individual. In some instances, excess biological sample from transducer module 2910 can be directed to an external (or alternatively internal) waste system 2908. In some instances, a cellular analysis system 2900 may include one or more features of a transducer module or blood analysis instrument such as those described in previously incorporated U.S. Pat. Nos. 5,125,737; 6,228,652; 8,094,299; and 8,189,187.

### Fluorescence System

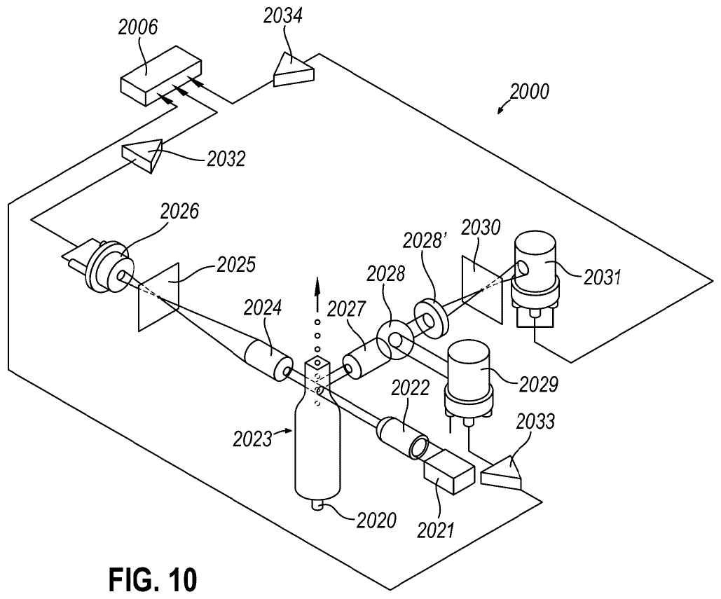

FIG. 10 depicts an illustrative flow cytometer 2000 that may be utilized in a fluorescence system in order to measure various parameters of a sample fluid as would be apparent to one skilled in the art in view of the teachings herein. In some instances, cells from a hematological sample are treated with a hemolytic agent to lyse erythrocytes, thereby leaving behind white blood cells in the sample fluid. Further, the remaining white blood cells may then be stained with a fluorescent dye which can make a difference in the fluorescence intensity. Such a preparation procedure may utilize the teachings of sample preparation process described herein. With the white blood cells suitably stained in accordance with the description herein, the sample fluid containing stained cells may be introduced into flow cytometer 2000 to measure scattered light and fluorescence of the respective cells when the cells are irradiated with a laser.

Flow cytometer 2000 includes a light source 2021 (e.g. a red semiconductor laser), configured to emit a beam of light (e.g. a laser beam with a wavelength of 633 nm) into an orifice part of a sheath flow cell 2023 via a collimating lens 2022. Simultaneously, particles from the sample fluid (e.g., cells-such as blood cells or body fluid cells) individually pass through nozzle 2020 into the orifice part of sheath flow cell 2023. Therefore, the particles are directed into the sheath fluid and configured to pass through an emitted beam of light from light source 2021 within sheath flow cell 2023. The light source 2021 irradiates an orifice part of a flow cell into which the prepared measuring sample has been introduced, with light which can excite a dye used in treatment of a sample, and is selected depending on a fluorescent dye which stains a particle (e.g., blood cell or body fluid cell) in a sample. Therefore, depending on a kind of a fluorescent dye used, in addition to the semiconductor laser, for example, an red argon laser, a He—Ne laser, and a blue semiconductor laser may be used.

Forward scattered light radiated from the particle is introduced into a forward scattered light detector 2026 (e.g., a photodiode) via a condensing lens 2024 and a pinhole plate 2025. Additionally, side scattered light radiated from the particle is introduced into a side scattered light detector 2029 (e.g., photomultiplier tube) via a condensing lens 2027 and a dichroic mirror 2028. Side fluorescent light radiated from the particle is also introduced into a side fluorescent light detector 2031 (e.g., photomultiplier tube) via condensing lese 2027, a dichroic mirror 2028, a filter 2028′ and a pinhole plate 2030. A forward scattered light signal outputted from the forward scattered light detector 2026, a side scattered light signal outputted from the side scattered light detector 2029, and a side fluorescent signal outputted from the side fluorescent light detector 2031 are amplified with amplifiers 2032, 2033, 2034, respectively, and are inputted into the control part 2006. Control part 2006 analyses these signals, and calculates received signal intensities. Control part 2006, or any other suitable components of a fluorescent system, may utilize these scattered light intensities in order to calculate and display suitable measured parameters, as would be apparent to one skilled in the art in view of the teachings herein. Further information on fluorescence systems which may be applied to cell analysis in some embodiments is provided in U.S. Pat. Nos. 7,625,730 and 7,892,841, the disclosures of each of which are hereby incorporated by reference in their entirety.

Note, the fluorescence systems are sometimes referred to as an optical system in the art, as they leverage laser excitation and the use of mirrors in a non-imaging arrangement, thus the fluorescence systems can also be referred to as an optical system.

Some fluorescence technologies may also leverage imaging as part of an analytical process (e.g., fluorescence in situ hybridization, aka FISH). A fluorescence imaging module (e.g., FISH) may be used as part of an additional module used to assess biological samples (e.g., blood cells) as a different module from the flow imaging modules described earlier. In this context, the use of fluorescence can apply to imaging or non-imaging systems or modules, as appropriate. For instance, a multi-module analysis system can include a flow imaging module (e.g., FIG. 1) and a fluorescent imaging module—as separate imaging modules. Alternatively, a multi-module analysis system can include a flow imaging module (e.g., FIG. 1) and a separate fluorescence module which may comprise a fluorescent imaging component. Alternatively, a multi-module analysis system can include an imaging module (e.g., flow imaging of FIG. 1 or FISH), and at least one separate module that does not utilize imaging (e.g., impedance, spectrophotometry, fluorescence cytometry, light scatter, or conductivity).

### Spectrophotometer System

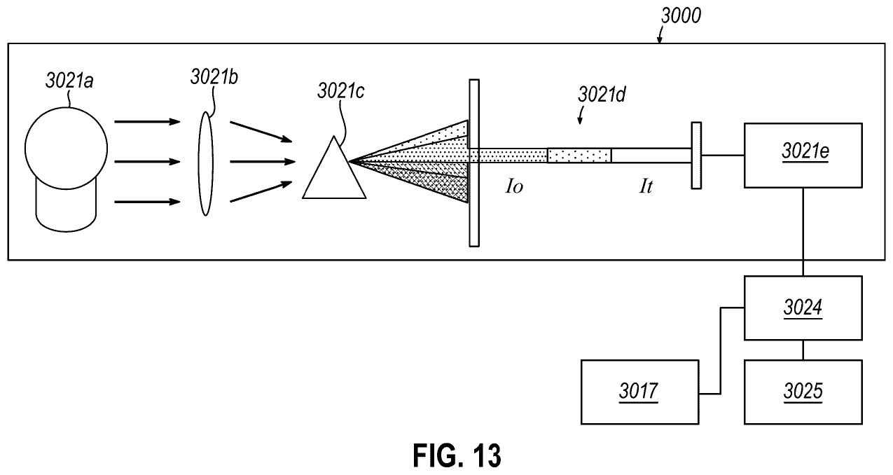

FIG. 13 shows a spectrophotometer 3000 operable to measure the absorption, transmittance, and/or other characteristic of a diluted and lysed blood sample- and used to measure red blood cell hemoglobin content—in one example, hemoglobin concentration for the blood sample. The measured characteristic is then converted into a corresponding measurement for the hematology parameter.

The spectrophotometer includes a light source 3021a, a lens 3021b, a prism 3021c, a cuvette 3021d, and a detector 3021e. To arrive at an absorption or transmittance reading, the blood sample is passed through the cuvette and the light source emits light through lens 3021b, prism 3021c, cuvette 3021d and the passing blood sample. Detector 3021e positioned on the opposite side of cuvette 3021d obtains an absorption and/or transmittance reading for the blood sample. To convert the absorbance and/or transmittance reading for the blood sample into a hematology measurement, a look up table may be used to correlate the reading to the hematology measurement, or alternatively the system is programmed to make this calculation. This is accomplished by a processor 3024 and memory 3025.

In the embodiment of FIG. 13, processor 3024 and memory 3025 are included as part of the automated hematology analyzer. However, processor 3024 and memory 3025 may also take a number of different forms, such as a processor in a connected personal computer or other instrument operable to convert the absorbance and/or transmittance reading into an uncorrected hematology measurement, such as hemoglobin concentration. In this embodiment, processor 3024 may be any commercially available microprocessor. Processor 3024 in association with the memory 3025 is further operable to take the uncorrected hematology measurement and convert it into a corrected hematology parameter, wherein the corrected hematology parameter is based on the uncorrected hematology measurement and the temperature measurement taken by the temperature sensor 3017. This corrected hematology measurement compensates for the inaccuracy in the uncorrected hematology measurement due to temperature and provides a more accurate measurement for the hematology parameter measured in the blood sample.

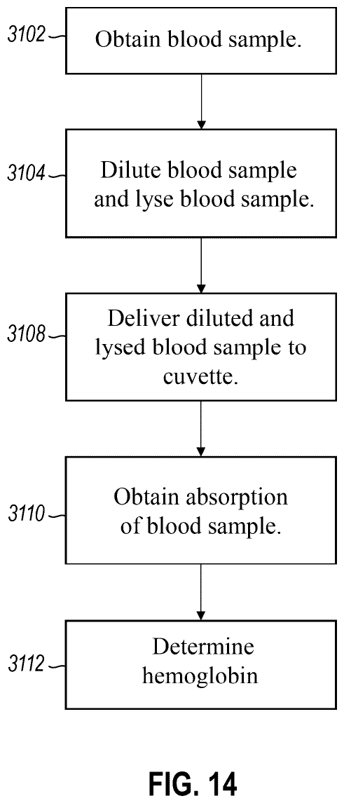

FIG. 14 shows that once the blood sample has been obtained 3102 and is diluted and lysed 3104, it is passed through cuvette 3021d in step 3108. As explained above, in one embodiment, cuvette 3021d is part of a spectrophotometer 3000 or other measurement instrument. The spectrophotometer 3000 obtains an absorption and/or transmittance measurement for the blood sample in step 3110. This measurement is then passed on to processor 3024 in step 3112, where a hemoglobin measurement is determined by processor 3024 based on the absorption/transmittance measurement. In one embodiment, processor 3024 determines the hemoglobin measurement using look up tables stored in memory 3025, or is programmed to correlate the absorption/transmittance measurement to a hemoglobin measurement. In particular, to arrive at the hemoglobin measurement, processor 3024 simply uses the absorption measurement obtained for the blood sample to arrive at a corresponding hemoglobin measurement. The processor may then obtain a hemoglobin measurement in step 3112.

### System Implementation

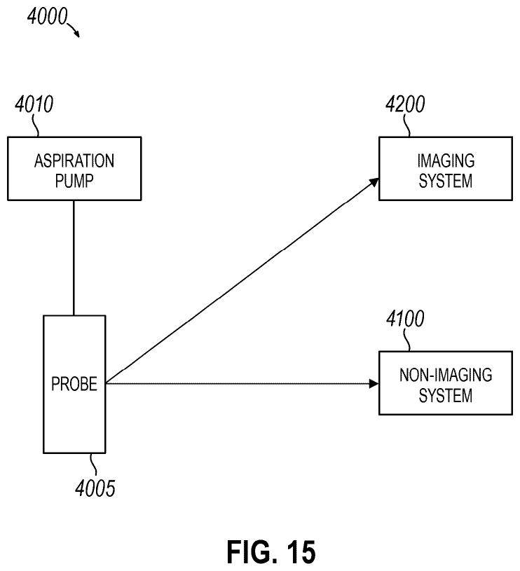

FIG. 15 shows an example of an imaging system and non-imaging system combined into one testing apparatus 4000. Testing apparatus 4000 may include a Sample Aspiration Module (SAM), an imaging system 4200, and a non-imaging system 4100 (e.g., an impedance system, conductivity system, light scatter system, or fluorescence system). SAM may include a probe 4005 and an aspiration pump 4010. Imaging system 4200 and non-imaging system 4100 may be in fluid communication with SAM such that SAM is capable of providing imaging system 4200 and non-imaging system 4100 with fluid samples. In other words, imaging system 4200 receives one portion (e.g., a first portion) of a blood sample and non-imaging system 4100 receives another portion (e.g., a second portion) of a blood sample (e.g., two different aliquots of the same blood sample, or an aliquot of the same blood sample divided into a first portion which goes into imaging system 4200 and a second portion which goes into non-imaging system 4100).

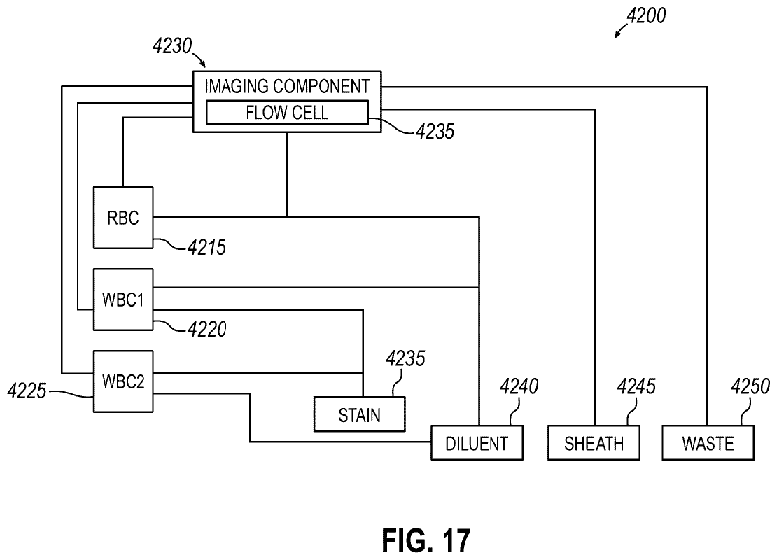

FIG. 17 shows a detailed example of an imaging system 4200 of testing apparatus 4000. Imaging system 4200 may include a RBC chamber 4215, a first WBC chamber 4220, a second WBC chamber 4225, an imaging component 4230 having a flowcell 4233, a stain 4235, a diluent 4240, a sheath 4245, and a waste container 4250. This is for illustrative purposes, and there can be any combinations of RBC chambers and WBC chambers. The blood is separated into RBC chambers and WBC chambers as the blood in the WBC chambers receives additional reagents and preparation, as will be explained herein.

Probe 4005 may be used to mix various fluid samples prior to use. Once mixed, the sample may be aspirated using vacuum at probe 4005 from aspiration pump 4010, probe may then be consecutively positioned into the RBC chamber 4215 and both of the WBC chambers 4220, 4225 to thereby deliver a first portion of blood sample to the RBC 4215 and WBC chambers 4220, 4225. In one embodiment, RBC chamber 4215 is configured to receive diluent while WBC chambers 4220, 4225 are configured to receive diluent, a lysing reagent (to lyse/remove red blood cells), and a staining reagent (to stain the nuclear region of the white blood cells). The divided blood samples in the WBC chambers 4220, 4225 may then be mixed with stain 4235 and diluent 4240 and incubated in chambers 4215, 4220, 4225 using integrated heaters. Due to the difficulty in differentiating white blood cells, it is helpful to stain the nucleus region to better show and display the nucleus region to aid in white blood cell differentiation (e.g., differentiating between at least neutrophils, lymphocytes, monocytes, eosinophils, and basophils). The lyse is used to eliminate red blood cells during this white blood cell analysis cycle.

In one embodiment, the staining and lysing reagents are two separate compounds adding during separate deposition steps. In one embodiment, the stain and lysing reagents are in one composition containing both a stain and a lyse together—where the composition includes saponin, a plurality of stains (e.g., combinations of new methylene blue, crystal violet, and basic fuchsin), and glutaraldehyde. Additional information on stain and lyse compositions can be found in U.S. Pat. No. 9,279,750 and U.S. published patent application 2021/0108994, the disclosures of each of which are incorporated herein by reference in their entirety.

Once incubated, the blood may be delivered to a flowcell within imaging component 4230 (e.g., 22 of FIG. 1). Blood from RBC chamber 4215 is imaged in one cycle. Note this cycle takes less time since the RBC chamber does not receive a stain and lyse reagent. Blood from WBC chambers 4220, 4225 is imaged in a different cycle (e.g., a separate two cycles). Once in flowcell 4233 and within a stream of sheath 4245, an Optical Bench Module (OBM) may capture cell images and convert the full frames to patches. After conversion, an Image Processing Module (IPM) may preprocess and classify the patches. The classified patches may then be used to generate analysis data.

The sample portions that remains in the chambers 4215, 4220, 4225 may then pass from their respective chambers to an alternative system (not shown), which can do further measurements on the sample (e.g., for different analytical tests). Alternatively, whatever sample portion remaining in chambers 425,4220, 4225 is flushed to a waste container 4250, and the chambers are cleaned (e.g., with diluent) in anticipation of receiving another blood sample. Portions of specimen already analyzed through imaging component 4230 and (optionally in an alternative system after the imaging step) may then be deposited to a waste container 4250 and imaging system 4200 cleaned (e.g., with diluent) in preparation for a subsequent blood sample.

The inclusion of a non-imaging system 4100 can be useful for various reasons, including to provide a secondary source of information using more traditional blood analysis techniques to confirm results, or to provide analysis for cell parameters that may be difficult to assess via imaging—for instance volumetric data such as mean corpuscular volume (MCV), or hemoglobin content of red blood cells. In some embodiments, system 4100 rather than a non-imaging system can be an alternative system that performs supplemental imaging in another way as an additional step to the flow-imaging system of imaging system 4200. In various examples, the non-imaging system can include various combinations of impedance, conductivity, light scatter, volume-conductivity-scatter (VCS), fluorescence, and spectrophotometry modules.

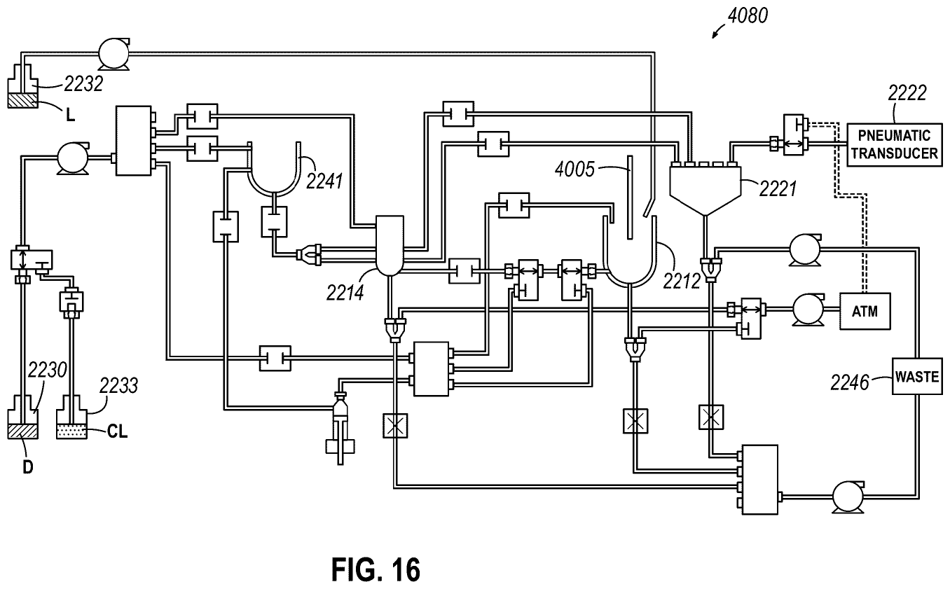

FIG. 16 shows non-imaging system 4100 that includes, among other components, a pair of fluid analysis chambers including a first fluid analysis chamber in the form of a first bath 2212 and a second fluid analysis chamber in the form of a second bath 2214. First bath 2212 is a white blood cell (WBC) or hemoglobin (HGB) bath and second bath 2214 is a red blood cell (RBC) bath. In the example shown the WBC bath 2212 is open for permitting a sample probe 4005 of the testing apparatus 4000 to selectively access the WBC bath 2212, such as to aspirate fluid therefrom and/or dispense fluid thereto. While not shown, the WBC and RBC baths 2212, 2214 of the present embodiment may be housed within the confines of non-imaging system 4100. Non-imaging system 4100 also includes a sweep tank 2241 in selective fluid communication with both baths 2212, 2214. The non-imaging system 4100 also includes a plurality of fluid reservoirs including a first fluid reservoir in the form of a diluent reservoir 2230 containing a diluent (D), a second fluid reservoir in the form of a lyse reservoir 2232 containing lyse (L), and a third fluid reservoir in the form of a cleaner reservoir 2233 containing a cleaner (CL).

Diluent reservoir 2230 is in fluid communication with sweep flow tank 2241, WBC bath (212), and RBC bath 2214. Further, cleaning reservoir 2233 is in fluid communication with sweep flow tank 2241, baths 2212, 2214, and any other suitable components as would be apparent to one skilled in the art in view of the teachings herein. Non-imaging system 4100 may deliver diluents (D) from diluents reservoir 2230 to sweep flow tank 2241, WBC bath 2212, and RBC bath 2214 in order to suitably dilute samples in accordance with the description herein. In some instances, sweep tank 2241 may selectively receive diluent (D) and cleaner (CL) in accordance with the description herein, and also communication such received fluids to baths 2212, 2214. It should also be understood that baths 2212, 2214 may also be in fluid communication with reservoirs 2230, 2233 such that baths 2212, 2214 may directly receive diluent (D) and cleaner (CL).

Non-imaging system 4100 is configured to suitably communicate cleaner (CL) baths 2212, 2214, sweep flow tank 2241, and various other suitable components of non-imaging system as would be apparent to one skilled in the art in view of the teachings herein. Cleaner (CL) may be distributed throughout system 4100 in order to suitably remove traces of previous samples processed by system 4100.

Further, lyse reservoir 2232 is in fluid communication with WBC bath 2212. Non-imaging system 4100 is configured to deliver lyse (L) from lyse reservoir 2232 into WBC bath 2212 in order to suitably lyse a blood sample to suitably remove red blood cells from the sample in WBC bath 2212.

Baths 2212, 2214 and/or sweep flow tank 2241 are also in suitable communication with sample analyzer 2221 such that sample fluid may be communicated to sample analyzer 2221 for suitable analysis as would be apparent to one skilled in the art in view of the teachings herein. A waste receptable 2246 is in fluid communication with various components of system 4100 such that processed sample, diluent (D), cleaner (CL), lyse (L), etc., that have been used in conjunction with system 4100 may be suitable disposed of after illustrative use.

The non-imaging system 4100 is configured to analyze a biological sample. In some embodiments, the non-imaging system 4100 is configured to analyze a blood sample, such that the non-imaging system 4100 may be referred to as a blood analysis system. While not shown, the WBC bath 2212 of the present embodiment may include a hemoglobin transducer configured to measure an amount of hemoglobin present in a fluid medium contained within the WBC bath 2212. For example, the hemoglobin transducer may include a light source (e.g., a filtered light source) and an optical sensor configured to receive optical signals emitted from the light source through the fluid medium contained within the WBC bath 2212. In some embodiments, the WBC and RBC baths 2212, 2214 may each be fluidly coupled to a suitable sample analyzer 2221 via corresponding input and output conduits equipped with respective valves for selectively conveying fluid media from one of the WBC or RBC baths 2212, 2214 to the suitable sample analyzer 2221 and/or for returning such fluid media from the sample analyzer 2221 to the WBC or RBC bath 2212, 2214. The sample analyzer 2221 may be configured to measure any suitable parameter of the fluid media received form each bath 2212, 2214 as would be apparent to one skilled in the art in view of the teachings herein (e.g., a complete blood count, etc.). In other embodiments, only one of the WBC or RBC baths 2212, 2214 (e.g., only the RBC bath 2214), may be fluidly coupled to the sample analyzer 2221. In the example shown, the sample analyzer (221) is also fluidly coupled to a pneumatic transducer 2222. While analysis (e.g., impedance-based counting, optical techniques, and/or imaging) of blood is shown and described herein, the biological analysis system 2210 may analyze (and optionally image) a variety of fluids including, but not limited to, other bodily fluids such as synovial fluid, urine, bone marrow, etc.

It should be understood that non-imaging system 4100 may include any other suitable components as would be apparent to one skilled in the art in view of the teachings herein. Therefore, suitable fluid lines, pumps, valves, multi-flow units, etc., may be readily incorporated into non-imaging system 4100.

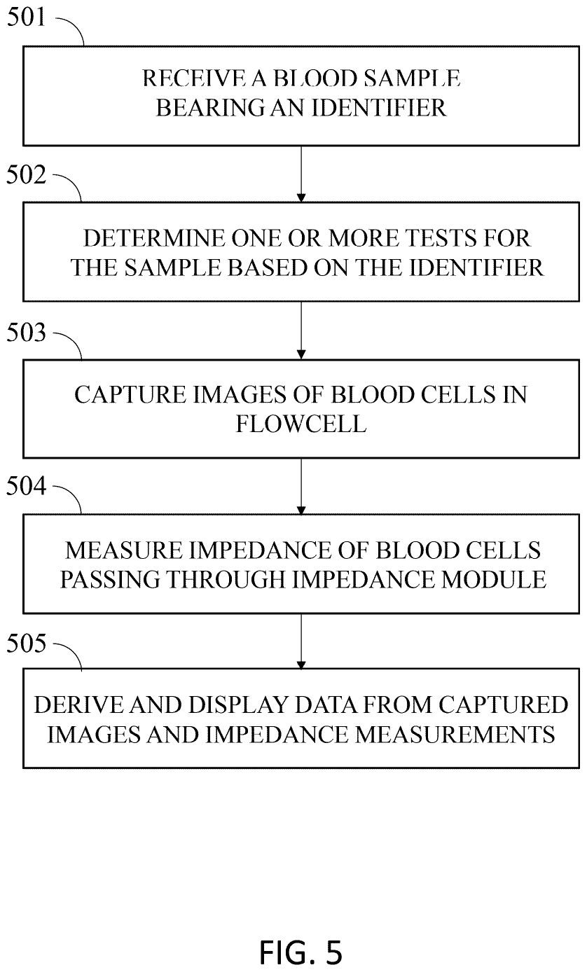

Referring back to FIG. 5, in some embodiments, a blood sample may be received in test tubes and/or obtained for testing that includes an identifier 501. For example, in some embodiments, the blood sample or blood sample container may include a barcode 4057, QR code, a Radio frequency identification (RFID), or the like. The identifier may contain relevant details about the sample, such as, for example, patient information, temporal data associated with the sample, desired testing procedure, and the like. Thus, in some embodiments, the system may automatically, or via user assistance, obtain the data contained in the identifier and determine 502 one or more tests for the sample.

Once the test is determined 502, the system may, in some embodiments, capture 503 images of blood cells in a flow cell. For example, a flow imaging system, such as shown in FIGS. 1, 1A, and 1B may be used to capture 503 images of blood cells as they pass through the flowcell. In addition to image capture 503, the system may also include an analysis system or transducer (e.g., 300 and 400) to measure 504 the impedance of blood cells (e.g., an alternative system). Other types of measurement channels or modules, such as fluorescence or spectrophotometry channels, may also be included. Using measurements from these various channels (e.g., captured images and measured impedance) data can be derived 505 related to the sample. This derived data may then be displayed 505 to a user or operate on for evaluation. By way of non-limiting example, Table 1, shown below, provides a non-exhaustive list of possible parameters that can be determined and/or derived regarding a sample using the disclosed technology.

TABLE 1

| Parameter | Description | Technology | Measurement |
|---|---|---|---|
| RBC | Red Blood Cell | Flow Imaging | Directly Measured |
| RBC-i | Red Blood Cell | Impedance | Directly Measured |
| HGB | Hemoglobin | Spectro- | Directly Measured |
| | | photometry | |
| HCT | Hematocrit | Impedance | Calculated |
| MCV | Mean Cell Volume | Impedance | Derived from RBC |
| | | | Histogram |
| MCH | Mean Corpuscular | Impedance | Calculated |
| | Hemoglobin | | |
| MCHC | Mean Corpuscular | Impedance | Calculated |
| | Hemoglobin Conc | | |
| RDW | Red Distribution | Impedance | Derived from RBC |
| | Width | | Histogram |
| RDW-SD | Red Distribution | Impedance | Derived from RBC |
| | Width SD | | Histogram |
| PLT | Platelet | Flow Imaging | Directly Measured |
| PLT-i | Platelet | Impedance | Directly Measured |
| MPV | Mean Platelet Volume | Impedance | Derived from PLT |
| | | | Histogram |
| WBC | White Blood Cell | Flow Imaging | Directly Measured |
| NRBC % | Nucleated Red Blood | Flow Imaging | Calculated |
| | Cell Percent | | |
| NRBC# | Nucleated Red Blood | Flow Imaging | Directly Measured |
| | Cell Number | | |
| OTHR % | Unclassified white | Flow Imaging | Calculated |
| | blood cells percent | | |
| OTHR# | Unclassified white | Flow Imaging | Directly Measured |
| | blood cells number | | |
| NE % | Neutrophil Percent | Flow Imaging | Calculated |
| NE# | Neutrophil Number | Flow Imaging | Directly Measured |
| IG % | Immature Granulocyte | Flow Imaging | Calculated |
| | Percent | | |
| IG# | Immature Granulocyte | Flow Imaging | Directly Measured |
| | Number | | |
| LY % | Lymphocyte Percent | Flow Imaging | Calculated |
| LY# | Lymphocyte Number | Flow Imaging | Directly Measured |
| MO % | Monocyte Percent | Flow Imaging | Calculated |
| MO# | Monocyte Number | Flow Imaging | Directly Measured |
| EO % | Eosinophil Percent | Flow Imaging | Calculated |
| EO# | Eosinophil Number | Flow Imaging | Directly Measured |
| BA % | Basophil Percent | Flow Imaging | Calculated |
| BA# | Basophil Number | Flow Imaging | Directly Measured |
| RET % | Reticulocyte Percent | Flow Imaging | Calculated |
| RET# | Reticulocyte number | Flow Imaging | Directly Measured |
| IRF | Immature Retics | Flow Imaging | Calculated |
| | Fraction | | |

For the illustrative purposes of Table 1, the majority of flow imaging derived cell data is associated with a cell count, and therefore the data derived from the images is primarily a count. In other examples, quantitative data on individual cell types can be associated with the flow imaging technology—for example, cell diameter or nuclear area of individual cells.

In some embodiments an aliquoter may be configured to separate the sample into a plurality of aliquots such that each aliquot may undergo a separate analysis (e.g., image based, or impedance based). Thus, it should be understood, as discussed herein, that the sample may be partitioned and passed to different modules for analysis. For example, in some embodiments, the analytic system may be adapted to flow a first portion of a sample through the flow imaging module for red blood cell (RBC) imaging while another portion of the sample is passed through a second flowcell for white blood cells (WBC) imaging.

### Non-Smear Based Image Analysis

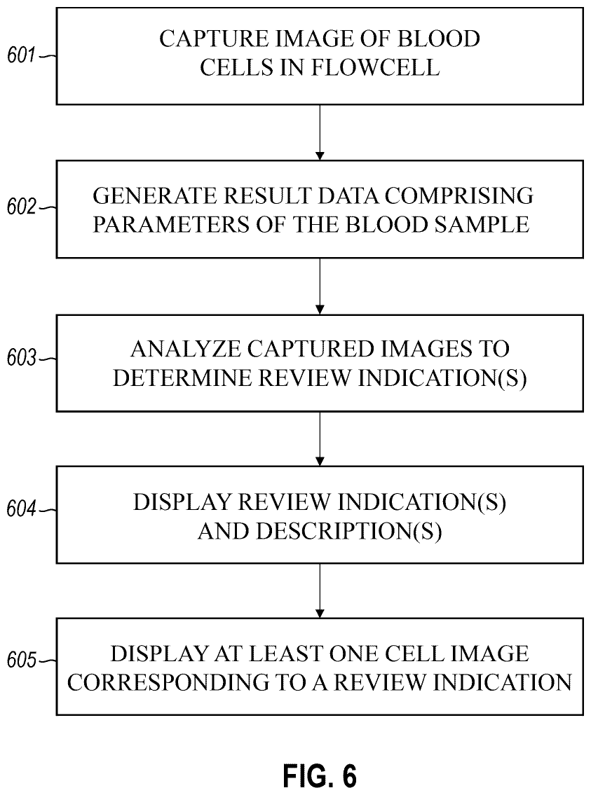

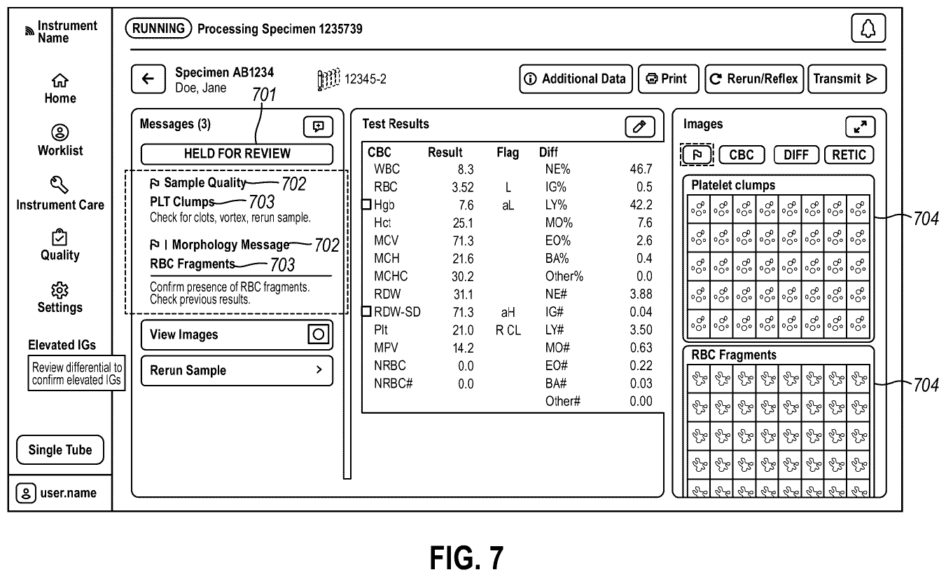

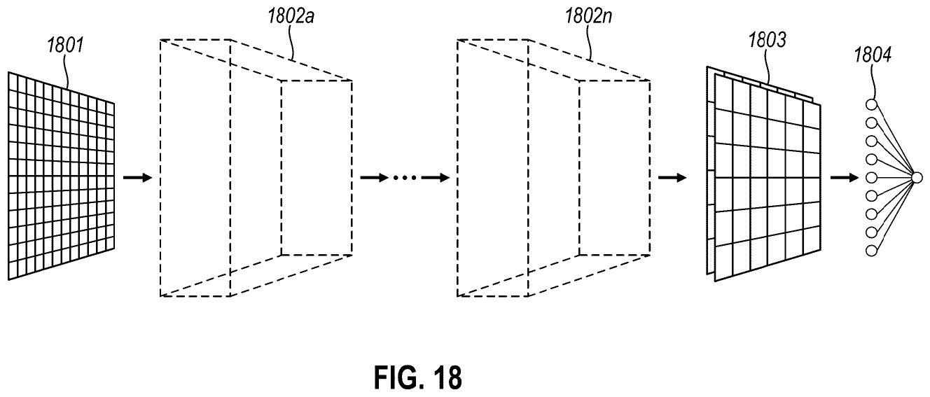

In a further embodiment, and as shown in FIGS. 6 and 7, the system may include a user interface that allows a user to evaluate potential outliers or errors in the analysis without requiring manual evaluation (e.g., a smear). In other words, a user can use the images derived from flow imaging on a screen to confirm a result without the need to do separate imaging utilizing a smear/slide sample-thereby saving considerable time. Alternatively, a user can use the images presented to confirm that cells are labelled correctly, and/or to confirm a presented result. In some cases, this type of functionality may be implemented using algorithms which would analyze captured images and/or related data such as impedance measurements, and identify issues which may require further review. An example of a method which may be implemented to allow a user to review such issues is illustrated in FIG. 6. In the method shown in that figure, initially, images may be captured 601 by an image capture device as they pass through a flow cell. Based on the captured 601 images, potentially as well as other types of data (e.g., impedance measurements, fluorescence measurements, etc.) a processor (e.g., 18) may be able to generate 602 result data comprising parameters of the sample (e.g., those shown in Table 1). The system may then analyze the captured images, potentially in combination with other data, to determine 603 review indications to present to a user. This may be done, for example, using a machine learning algorithm, such as that shown in FIG. 18 which has been trained to classify images of particles from a blood cell into various cell classifications, including normal and abnormal cell types. Note, FIG. 18 is provided as an illustrative example of an architecture for a cell classifier which is used to label cells, and various types of models for this purpose can be used such as neural networks, convolutional neural networks, modified publicly available neural networks. Additional examples can leverage pixel analysis and masking techniques to determine a cell classification. Further information on techniques which some embodiments may use in cell classification can be found in U.S. Pat. No. 11,403,751, the disclosure of which is hereby incorporated by reference in its entirety.

In various embodiments, a single classifier is used to classify all cell types, including abnormal cell types. In some embodiments, a plurality of classifiers may be used with a voting protocol used to provide a final classification of a cell type. In some embodiments, a plurality of classifiers includes a classifier specifically assigned to abnormal cell types or a subset of abnormal cell types (e.g., if a cell is classified as a red blood cell, that can trigger use of a classifier unique to identifying abnormal cell types associated with red blood cells).

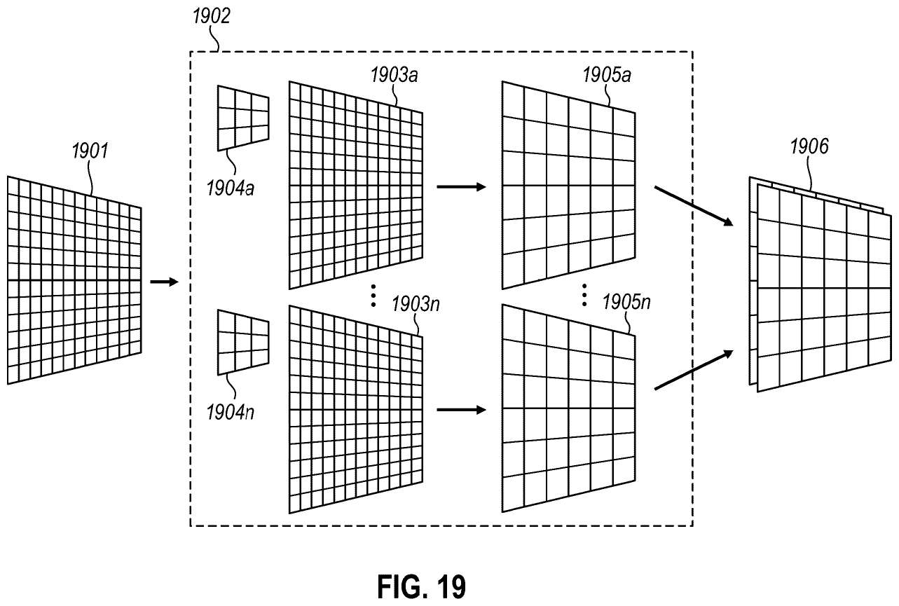

In the architecture of FIG. 18, an input image 1801 would be analyzed in a series of stages 1802a-1802n, each of which may comprise one or more layers, and which is illustrated in more detail in FIG. 19. As shown in FIG. 19, an input 1901 (which, in the initial layer 1902a of FIG. 18 would be a cell image and otherwise would be the output of the preceding stage) is provided to a stage 1902 where it would be processed by a convolutional layer of the stage 1902 to generate one or more transformed images 1903a-1903n. This processing may include convolving the input 1901 with a set of filters 1904a-1904n, each of which would identify a type of feature from the underlying image that would then be captured in that filter's corresponding transformed image. For instance, as a simple example, convolving an image with the filter shown in table 2

TABLE 2
Example convolution filter.

| | | |
|---|---|---|
| −1 | −1 | −1 |
| −1 | 8 | −1 |
| −1 | −1 | −1 |

could generate a transformed image capturing edges from the input 1901.

As shown in FIG. 19, in addition to generating transformed images 1903a-1903n a stage may also comprise a pooling layer that generates a pooled image 1905a-1905n for each of the transformed images 1903a-1903n. This may be done, for example, by organizing the appropriate transformed image into a set of regions, and then replacing the values in that region with a single value, such as the maximum value for the region or the average of the values for the region. The result would be a pooled image whose resolution would be reduced relative to its corresponding transformed image based on the size of the regions it was split into (e.g., if the transformed image 1903a-1903n had N×N dimensions, and it was split into 2×2 regions, then the pooled image 1905a-1905n would have size (N/2)×(N/2)). These pooled images 1905a-1905n could then be combined into a single output image 1906, in which each of the pooled images 1905a-1905n is treated as a separate channel in the output image 1906. This output image 1906 can then be provided as input to the next stage 1902a-1902n as shown in FIG. 18.

Returning to the discussion of FIG. 18, after a final output image 1803 has been created through the various stages 1802a-1802n of processing, the final output image 1803 could be provided as input to a fully connected layer that processes the output images and classifies the input image into one of a plurality of categories. The plurality of categories may comprise, e.g. consist of, various types of images which may be captured (e.g., WBCs, RBCs) including types of images whose presence may trigger a review indicator (e.g., platelet clumps).

Exemplarily, the trained CNN may comprise the following layers:
i. An input layer that receives an 128×128×3 RGB image depicting a red blood cell image, immediately followed by
ii. A convolutional layer with 64 5×5 filters and the ReLU activation function, immediately followed by
iii. A 2×2 max pooling that generates a 64×64×64 output, immediately followed by
iv. A convolutional layer with 128 5×5 filters and the ReLU activation function, immediately followed by
v. A 2×2 max pooling that generates a 32×32×128 output, immediately followed by
vi. A convolutional layer with 256 5×5 filters and the ReLU activation function, immediately followed by
vii. A 2×2 max pooling that generates a 16×16×256 output, immediately followed by
viii. A convolutional layer with 512 5×5 filters and the ReLU activation function, immediately followed by
ix. A 2×2 max pooling that generates a 8×8×512 output, immediately followed by
x. A convolutional layer with 512 5×5 filters and the ReLU activation function, immediately followed by
xi. A 2×2 max pooling that generates a 4×4×512 output, immediately followed by
xii. A fully connected layer that generates a K scalar values, wherein K is the number of categories into which the cell images are classified. For instance, if the NN is trained to classify cell images into one of the front facing and not-front facing category, K is equal to two. For example, if the NN is trained to classify cell images into one of figure categories, K is equal to five.

These classifications may then be compared to thresholds (e.g., expected percentages or numbers of the particular particle types) and, if one or more thresholds were exceeded (or, in the case of low thresholds, not met), a system implemented based on this disclosure may determine 603 that corresponding review indication(s) (e.g., flags) should be presented to a user. For instance, if an abnormal cell type exceeds a particular percentage (illustratively, if RBC fragments exceed a 2.5% threshold) then it is flagged as abnormal—or alternatively if an abnormal cell type exceeds a particular count in a blood sample (illustratively, more than three blasts) then it is flagged as abnormal. These counts or particular percentages can be based on customized programmed rules, rules set up by a user, or rules derived from practical lab standards. These review indications may also be provided along with descriptions indicating, in the case of abnormal particle types, the abnormal particle type that triggered the indication. These review indications are particularly helpful to point out the abnormal particle types to a user, allow them to review any associated abnormal particle images on a screen without need to conduct a follow up confirmation test (e.g., a smear), and help confirm the abnormal particle type.

There are potentially several types of scores associated with cells as they are classified. For instance, a cell would have to exceed a certain classification threshold to be labelled as a first cell type (e.g., platelet), then an additional classification threshold to be labelled as an abnormal cell type (e.g., a giant platelet), and finally a particular numerical threshold would need to be exceeded for a review indication associated with the abnormal cell type (e.g., a flag for giant platelet) to be cited. Illustratively, an imaged cell may need to exceed a 60% confidence score to be assigned as a platelet, a 50% confidence score to be assigned as a giant platelet (or alternatively, once assigned as a platelet it is sent to a subclassifier and that subclassification would need to exceed a particular threshold—e.g., 70% to be assigned as a giant platelet), and then the overall number of giant platelets would need to exceed a numerical threshold (e.g., 2.5%) in order for a sample to be flagged for giant platelet. Please note, these are illustrative examples and any range of confidence scores and numerical thresholds can be used, and it is likely that difference confidence scores and different numerical thresholds may be used for different cell types.

Additionally, a review indication of an abnormal cell type may be different from an image review of an abnormal cell type. For instance, all giant platelets may be viewable as a separate category of images unique to those cell types (e.g., a giant platelet cell category with associated images of giant platelets). However, in order to trigger a review indication (sample flagged for having an abnormally high number of Giant Platelets)—a particular threshold score for that indication (e.g., 2.5%) would need to be exceeded.

Examples of such abnormal cell types along with corresponding descriptions are provided below in table 3.

TABLE 3

| | | Base | | Measurement | |
|---|---|---|---|---|---|
| Specimen | Panel | Parameter | Description | Technology | Priority |
| Whole Blood | CBC | RBC | RBC Frag/Micro | Impedance | 1 |
| | | | RBC Fragments | Flow Imaging | |
| Whole Blood | | RBC | Sickled Cells | Flow Imaging | 1 |
| Whole Blood | | RBC | Dimorphic Reds | Impedance | 1 |
| Whole Blood | | RBC | Red Cell Agglutination | Impedance | 1 |
| | | | Red Cell Clumping | Flow Imaging | |
| Whole Blood | | PLT | Large Platelets | Flow Imaging | 1 |
| Whole Blood | | PLT | Giant Platelets | Impedance | 1 |
| Whole Blood | | PLT | Giant Platelets | Flow Imaging | 1 |
| Whole Blood | | PLT | Platelet Clumps | Impedance | 1 |
| Whole Blood | | PLT | Platelet Clumps | Flow Imaging | 1 |
| Whole Blood | DIFF | WBC | Variant LY | Flow Imaging | 1 |
| Whole Blood | | WBC | WBC Blasts | Flow Imaging | 1 |
| Whole Blood | Retic | RET | Reticulated RBC | Flow Imaging | 1 |

What review indications may be determined, and how they would be determined, may be based on the characteristics of the particular implementation, such as what data is gathered regarding a sample. To illustrate, consider a system in which both images and impedance are used to identify platelets, with platelet identifications based on images designated by PLT, and platelet identifications based on impedance being designated by PLT-i, for convenience. In such a case, the platelet results generated using imaging technology may be the primary parameters for reporting purposes (e.g., displayed on results screens with other parameters, while PLT-i results may only be available through lower level screens), and both the PLT and PLI-i results may be used to determine whether to provide a notification and accompanying description to the user based on logic such as that set forth below in table 4.

TABLE 4

| PLT | PLT-i | | |
|---|---|---|---|
| Flag | Flag | Quality Check | Reporting Status |
| N | N | Passes Internal | Unflagged PLT and PLT-i results; PLT |
| | | Quality Check | result reported |
| Y | N | Internal Quality | R (i.e., low confidence) flag attached |
| | | Check not applied | to PLT result; user replaces PLT with |
| | | | PLT-i value at the LIS |
| N | Y | Internal Quality | R (i.e., low confidence) flag attached to |
| | | Check not applied | PLT-i result, PLT result reported |
| Y | Y | Internal Quality | R (i.e., low confidence) flag attached to |
| | | Check not applied | both PLT and PLT-i result |
| N | N | Fails Internal | Flag both PLT and PLT-i results with R |
| | | Quality Check | (i.e., low confidence) |

An example of another approach which may be taken, either in addition to or as an alternative to that described in the context of table 4, would be to determine flags based on confidence or test result value. For instance, in some cases an analyzer may be configured with a built in confidence threshold, and results which are generated with confidence lower than this threshold maybe accompanied by a confidence flag indicating that they are low confidence and may need additional review. As another example, in some cases a user of an analyzer may be allowed to define various range limits, such as reference limits, action limits, and critical limits. In such cases, when a result is outside of the specified limit range, it may be provided with a flag indicating the limits it falls outside of.

In any case, once the results have been determined, an interface which may include various parameters and/or review indications and corresponding descriptions derived from the images, impedance or other data related to the sample may be displayed 604. An example of such an interface is shown in FIG. 7. In the interface shown in that figure, the user is presented with a worklist 701 comprising a set of review indications 702 and descriptions 703 of those review indications. The interface of FIG. 7 also provides the user with categorizations for the different review indications (i.e., “Sample Quality” and “Morphology Message”) and brief instructions for the types of review and/or other remedial actions which may be appropriate in light of the review indications which are displayed. To assist with this review, the interface of FIG. 7 displays 605 sets of thumbnail cell images 704 corresponding to the images which would be reviewed based on the review indicators. For example, in a case where a description for a review indicator states that platelet clumps were detected in the sample, a set of thumbnail cell images could be presented displaying thumbnails of images where platelet clumps were detected. These images may be presented in an order based on their contribution to the corresponding review indication (e.g., platelet clump images may be sorted in order of the size of the depicted clumps, or the confidence with which the clumps were identified), and when a thumbnail image is clicked on or otherwise selected, a full resolution copy of the image corresponding to the selected thumbnail could be displayed so that the user could perform the appropriate review tasks.

Variations on the above examples are also possible in terms of how review indications and thumbnail cell images may be presented. For example, in some cases, rather than displaying sets of thumbnail cell images corresponding to items in a worklist, a user may be provided with a list of parameters and corresponding review notifications and, in response to selecting a notification (or its corresponding parameter), may be provided with a set of thumbnail cell images for that parameter specifically. As another example of potential variations which may exist in some implementations, there are different approaches to presenting thumbnail cell images. For example, such thumbnail cell images may be presented in an order which is sorted according to factors such as capture order, size, shape, standard deviation from a mean, and the like. It is also possible that, in some cases, review indications may be provided that would not be associated with particular images. For example, if a non-imaging modality (e.g., impedance) identified a particular unexpected cell type in a sample, then a review indication may be provided with information indicating that a reflex test for the unexpected cell type should be run, but may not be accompanied by (or associated with) thumbnail cell images such as described above.

Other types of variations beyond those in the presentation of review indications and thumbnail cell images are also possible. To illustrate, consider potential review indications which may be provided not based on abnormal cell types, but based on results (e.g., counts) obtained for cells which would be expected to be present in a sample (e.g., red blood cells in a whole blood sample). An example of this type of illustration may be a low confidence flag, which some implementations may provide in the event that the confidence determined for a particular count (e.g., red blood cell count) is below a built in threshold for the analyzer which determined the count. In this case, a particular low confidence review indication (e.g., a flag having a different appearance from a flag that might be displayed for platelet clumps, or a different type of symbol entirely) may be displayed, and a user may be allowed to view thumbnails of the cell images corresponding to the low confidence review indicator (e.g., images which were identified as red blood cells with confidence below the threshold). As another example, in a case where a count exceeded a built in threshold corresponding to the level for which an analyzer claimed to be accurate (e.g., the analyzer claimed to be able to accurately count a cell type up to X, and a count of X+Y of that cell type was detected), a linearity review indication may be provided, along with thumbnail cell images of the cell type whose count exceeded the threshold, and a message indicating that the sample should be rerun after dilution.

As an example of yet another type of variation, in some cases, users may be able to specify one or more thresholds which should be applied to various counts for triggering review indications. For example, a user may define a first set of high and low thresholds for a cell type, and a second set of high and low thresholds for that cell type. In this case, if the count for that cell type was outside of the first set of high and low thresholds but not outside the second set of high and low thresholds, a review indication with a first characteristic may be provided (e.g., a flag colored yellow), while if the count for that cell type was outside of the second set of high and low thresholds, a review indication with a second characteristic (e.g., a flag colored red) may be provided. Accordingly the examples of review indications and their potential triggers provided above should be understood as illustrative only, and should not be treated as limiting on the scope of protection provided by this document or any other document which claims the benefit of this document.

### Multiple Channel System

As discussed herein, a sample may be partitioned (e.g., divided into aliquots) to allow for various types of testing. Thus, in some embodiments, the sample analysis system may include an aliquoter configured to separate samples into aliquots, wherein the controller (e.g., processor) is programmed to cause the fluidics system to control the flow of aliquots based on the parameters that need determined values.

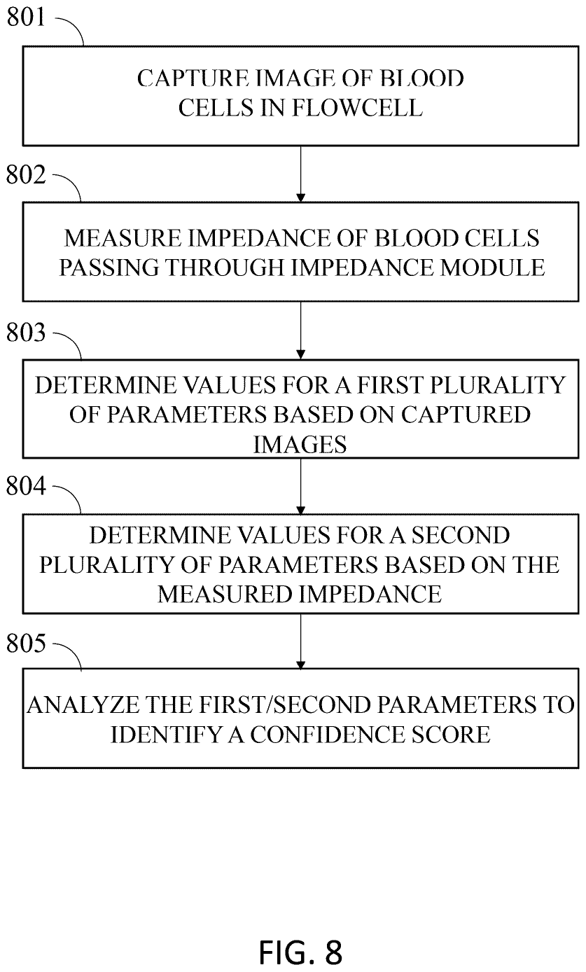

Referring now to FIG. 8, an illustrative flow diagram is shown for a dual channel system. As will be described in greater detail below, a dual channel system may be configured to capture high quality images of microscopic particles of a first aliquot of sample fluid (e.g., blood cells) in a flow cell via an imaging system in accordance with the description above, as well as analyze a second aliquot of the same sample fluid via a suitable alternative system in accordance with the description herein. In some embodiments, and as shown, the system may capture 801 images of blood cells in a flow cell (e.g., of an aliquot) and measure 802 the impedance of blood cells passing through an alternative system. Therefore, in the current illustrative example, dual channel system includes an imaging system in accordance with the description above, as well as an impedance system in accordance with the description above. Note, additional embodiments can use more than two channels—for instance, adding any of a spectrophotometry channel, a fluorescence channel, a conductivity channel, a light scatter channel, or a VCS channel. Though the term channel is used, the term can also be used synonymously with module and is meant to signify the use of a different analytical process to analyze particles—is this concept each channel or module uses a different analytical technique for particle analysis (e.g., an imaging technique different from an impedance technique, in turn differing from a spectrophotometry technique).

While the illustrative example shown in FIG. 8 describes measuring 802 impedance of blood cells passing through an alternative system, it should be understood that blood cells of the sample fluid may be analyzed using alternative systems which may not measure impedance, such as fluorescence image analyzing apparatus 2001 and/or spectrophotometer system (3000) described above. It should also be understood that, while this illustrative example is described in terms of a channel for an imaging system and a channel for an alternative system, any number of channels using any types of differing measurement systems (e.g., fluorescence, light scatter and/or spectrophotometry systems) may be included in various implementations. Therefore, it should be understood that multi-channel systems (including, but not limited to, dual channel systems) may utilize the imaging system with flow cell 22, high optical resolution imaging device 24, and processor 18 in order to capture images from a first aliquot of sample fluid and that other channels of a multi-channel system may include any other suitable system configured to suitably analyze other aliquots of sample fluid.

Once the images are captured 801 and the impedance measured 802, the system may utilize an analysis module to determine values 803 for a first plurality of parameters using data from the flow imaging module and determine values 804 for a second plurality of parameters using data from the alternative system (or any other suitable alternative system as would be apparent to one skilled in the art in view of the teachings here). As an example, the system may determine 803 one or more image-based numerical values based on an analysis of the captured 801 images of blood cells, and determine 804 one or more numerical parameters based on measurements 802 from the alternative system (e.g., impedance system).

The first and second parameters may then be analyzed 805 to identify a confidence score or review indication. The first and second parameter may be analyzed 805 for any other suitable purpose as would be apparent to one skilled in the art in view of the teachings herein. Additionally, the system may present the determined values 803, 804 (which may include the one or more image-based numerical values and as well as the one or more numerical parameters based on measurements 802 of alternative system) to a user via a computing interface.

In some instances, at least one of the first measured parameters from the imaging system described above, and at least one the second parameter measured from the suitable alternative system of a multi-channel system (e.g., two channel system, or two channels within a more than two-channel arrangement) are similar and/or the same. The similar and/or matching measured parameters from the imaging system and the alternative system of the multi-channel system may be utilized by the multi-channel system for any suitable purpose as would be apparent to one skilled in the art in view of the teachings herein.

In a further embodiment, the first parameters (e.g., the parameters associated with the captured images) may include, but is not limited to: nucleated red blood cell percent, nucleated red blood cell number, unclassified white blood cell percent, unclassified white blood cell number, neutrophil percent, neutrophil number, immature granulocyte percent, immature granulocyte number, lymphocyte percent, lymphocyte number, monocyte percent, monocyte number, eosinophil percent, eosinophil number, basophil percent, basophil number, reticulocyte percent, reticulocyte number, and immature reticulocyte fraction. In another embodiment, the second parameters (e.g., the parameters associated with the measured impedance values) may include, but are not limited to, mean cell volume, mean corpuscular hemoglobin, mean corpuscular hemoglobin concentration, red cell distribution width, standard deviation of red cell distribution width, and mean platelet volume.

Unclassified cells refer to cells that fail to exceed a particular classification threshold to be assigned as a cell type. In various examples, the unclassified cells can be placed into a general unclassified classification bucket, where the images are presented for review by a user (e.g., to manually label/classify these cells on screen). Cells labeled as unclassified white blood cells may be classified as a white blood cell (e.g., exceed a first confidence threshold to be classified as a white blood cell) but fail to meet a confidence threshold to be classified as a specific type of white blood cell (e.g., one in the 5 or 6-part WBC differential).

### Processing Architecture

Turning next to FIG. 9, that figure is a simplified block diagram of an exemplary module system which could be used for performing various logic and/or controlling various components described herein. Module system 900 may be part of or in connectivity with a cellular analysis system. Module system 900 is well suited for producing data or receiving input related analysis. In some instances, module system 900 includes hardware elements that are electrically coupled via a bus subsystem 902, including one or more processors 904, one or more input devices 906 such as user interface input devices, and/or one or more output devices 908 such as user interface output devices. In some instances, system 900 includes a network interface 910, and/or a diagnostic system interface 940 that can receive signals from and/or transmit signals to a diagnostic system 942. In some instances, system 900 includes software elements, for example shown here as being currently located within a working memory 912 of a memory 914, an operating system 916, and/or other code 918, such as a program configured to implement one or more aspects of the techniques disclosed herein.

In some embodiments, module system 900 may include a storage subsystem 920 that can store the basic programming and data constructs that provide the functionality of the various techniques disclosed herein. For example, software modules implementing the functionality of method aspects, as described herein, may be stored in storage subsystem 920. These software modules may be executed by the one or more processors 904. In a distributed environment, the software modules may be stored on a plurality of computer systems and executed by processors of the plurality of computer systems. Storage subsystem 920 can include memory subsystem 922 and file storage subsystem 928. Memory subsystem 922 may include a number of memories including a main random-access memory (RAM) 926 for storage of instructions and data during program execution and a read only memory (ROM) 924 in which fixed instructions are stored. File storage subsystem 928 can provide persistent (non-volatile) storage for program and data files and may include tangible storage media which may optionally embody patient, treatment, assessment, or other data. File storage subsystem 928 may include a hard disk drive, a floppy disk drive along with associated removable media, a Compact Digital Read Only Memory (CD-ROM) drive, an optical drive, DVD, CD-R, CD RW, solid-state removable memory, other removable media cartridges or disks, and the like. One or more of the drives may be located at remote locations on other connected computers at other sites coupled to module system 900. In some instances, systems may include a computer-readable storage medium or other tangible storage medium that stores one or more sequences of instructions or code which, when executed by one or more processors, can cause the one or more processors to perform any aspect of the techniques or methods disclosed herein. One or more modules implementing the functionality of the techniques disclosed herein may be stored by file storage subsystem 928. In some embodiments, the software or code will provide protocol to allow the module system 900 to communicate with communication network 930. Optionally, such communications may include dial-up or internet connection communications.

It is appreciated that system 900 can be configured to carry out, or to cause a system to carry out, various aspects of methods such as described herein. For example, processor component 904 can be a microprocessor control module configured to receive cellular parameter signals from a sensor input device or module 932, from a user interface input device 906, and/or from a diagnostic system 942, optionally via a diagnostic system interface 940 and/or a network interface 910 and a communication network 930. Processor component 904 can also be configured to transmit cellular parameter signals, optionally processed according to any of the techniques disclosed herein, to sensor output device or module 936, to user interface output device 908, to network interface device 910, to diagnostic system interface 940, or any combination thereof. Each of the devices or modules described herein can include one or more software modules on a computer readable medium that is processed by a processor, or hardware modules, or any combination thereof.

User interface input devices 906 may include, for example, a touchpad, a keyboard, pointing devices such as a mouse, a trackball, a graphics tablet, a scanner, a joystick, a touchscreen incorporated into a display, audio input devices such as voice recognition systems, microphones, and other types of input devices. User input devices 906 may also download a computer executable code from a tangible storage media or from communication network 930, the code embodying any of the methods or aspects thereof disclosed herein. It will be appreciated that terminal software may be updated from time to time and downloaded to the terminal as appropriate. In general, use of the term “input device” is intended to include a variety of conventional and proprietary devices and ways to input information into module system 900.

User interface output devices 906 may include, for example, a display subsystem, a printer, a fax machine, or non-visual displays such as audio output devices. The display subsystem may also provide a non-visual display such as via audio output devices. In general, use of the term “output device” is intended to include a variety of conventional and proprietary devices and ways to output information from module system 900 to a user. Bus subsystem 902 provides a mechanism for letting the various components and subsystems of module system 900 communicate with each other as intended or desired. The various subsystems and components of module system 900 need not be at the same physical location but may be distributed at various locations within a distributed network. Although bus subsystem 902 is shown schematically as a single bus, alternate embodiments of the bus subsystem may utilize multiple busses.

Network interface 910 can provide an interface to an outside network 930 or other devices. Outside communication network 930 can be configured to effect communications as needed or desired with other parties. It can thus receive an electronic packet from module system 900 and transmit any information as needed or desired back to module system 900. As depicted here, communication network 930 and/or diagnostic system interface 942 may transmit information to or receive information from a diagnostic system 942. In addition to providing such infrastructure communications links internal to the system, the communications network system 930 may also provide a connection to other networks such as the internet and may comprise a wired, wireless, modem, and/or other type of interfacing connection. It is also possible that a network interface 910 may allow one module system to interface with one or more other systems to collectively provide functionality such as that described herein. For example, in some cases, a first module system which is local to an analyzer may control the analyzer, coordinate its various components and gather data regarding a sample, while a second module system which is located remotely (e.g., a cloud system separated from the first module system via a wide area network) may receive data from the first module system and analyze it to provide results such as could be provided on a user interface output device 908 of the first module system.

It will be apparent to the skilled artisan that substantial variations may be used in accordance with specific requirements. For example, customized hardware might also be used and/or particular elements might be implemented in hardware, software (including portable software, such as applets), or both. Further, connection to other computing devices such as network input/output devices may be employed. Module terminal system 900 itself can be of varying types including a computer terminal, a personal computer, a portable computer, a workstation, a network computer, or any other data processing system. Due to the ever-changing nature of computers and networks, the description of module system 900 depicted in FIG. 9 is intended only as a specific example for purposes of illustration. Many other configurations of module system 900 are possible having more or less components than the module system depicted in FIG. 9. Any of the modules or components of module system 900, or any combinations of such modules or components, can be coupled with, or integrated into, or otherwise configured to be in connectivity with, any of the cellular analysis system embodiments disclosed herein. Relatedly, any of the hardware and software components discussed above can be integrated with or configured to interface with other medical assessment or treatment systems used at other locations.

### Example of Sample Preparation Process

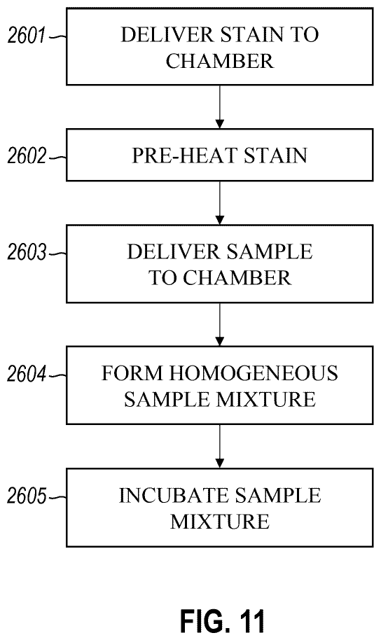

In systems described herein, a process such as shown in FIG. 11 may be used to perform sample preparation prior to sample fluids being analyzed in accordance with the description herein. Initially, in the process of FIG. 8, the staining agent may be delivered to a chamber, such as the mixing chamber, RBC chamber 4015, and/or WBC chambers 4020, 4025, ss described herein, at step 2601. This may comprise, for example, delivering the staining agent to the chamber via a stain dispenser. The staining agent may then be pre-heated within the chamber such as via induction heating, at step 2602. Next, the sample may be delivered to the chamber at step 2603. This may comprise, for example, delivering the sample to the chamber through a sample dispenser (e.g., probe 4005) so as to be added to the staining agent. In some embodiments, the delivery of the sample to the chamber may include mixing of the sample with the pre-heated stain within the chamber. In the process of FIG. 11, a homogeneous sample mixture may then be formed within the chamber at step 2604. This may comprise, for example, using fluid energy to mix the sample with the stain, such as by cyclically pulling the sample out of and pushing the sample back into the chamber via a corresponding tangential port of the housing to perform a regurgitative mixing. Alternatively, this may comprise using a magnet to drive a spherical ferromagnetic ball placed within the chamber to perform an agitative mixing. As another example, this may comprise introducing one or more bubbles at a bottom of the chamber to create a vortex.

The homogenous sample mixture may then be heated within the chamber such as via induction heating or resistive heating, at step 605. In some embodiments, the homogeneous sample mixture may be heated to a threshold temperature via induction heating or resistive heating, and may then be maintained at the threshold temperature via a maintenance heater.

After the homogenous sample mixture reaches the threshold temperature, the sample mixture may be conveyed to a flowcell, such as the flow cell 22 of FIG. 1 for being imaged by a camera such as the high optical resolution imaging device 24 of FIG. 1.

While the formation and induction heating of the sample mixture has been described as occurring within the chamber, it will be appreciated that alternative arrangements may include a tubing having a lumen (not shown) in which the sample mixture may be formed and induction heated in manners similar to those described above. In addition, or alternatively, any one or more of the teachings herein may be combined with any one or more of the teachings disclosed in U.S. Pat. No. 9,429,524, entitled “Systems and Methods for Imaging Fluid Samples,” issued on Aug. 30, 2016, the disclosure of which is hereby incorporated by reference in its entirety.

In some embodiments, the addition of diluent is part of the preparation step, where the diluent is added to each chamber during before, after, or both before and after a blood sample is added to each chamber. For example, an RBC chamber can receive diluent as the primary or sole preparation reagent, while a WBC chamber can receive both diluent and stain.

It should be appreciated that the preparation step for RBC chambers can be different than WBC chambers. For instance, the RBC chambers would utilize a preparation step involving: a) receiving a diluent followed by a blood sample, b) receiving a blood sample followed by a diluent, or c) receiving a diluent, followed by a blood sample, followed by additional diluent; but would not receive a stain. In this way, the preparation time for the RBC chambers may be shorter and a workflow can involve running an RBC sample through an imaging process while the WBC samples are still being prepared.

In some embodiments, a staining reagent utilizes both a lysing agent (to lyse red blood cells) and a staining agent (to permeate the remaining white blood cells, stain the interior region, and repair the white blood cell so stain does not escape). In this way, a single staining reagent can be used to process certain types of cells (e.g., white blood cells) to both eliminate red blood cells and stain the remaining white blood cells. Other embodiments can utilize a plurality of compositions, for instance a first lysing reagent to lyse red blood cells, and a second staining reagent to stain white blood cells, where a workflow would involve a chamber (e.g., a WBC chamber) receiving a separate lysing reagent and a separate staining reagent to prepare WBC samples for visualization.

In some embodiments, the various chambers (e.g., RBC chamber 4015, and WBC chambers 4020, 4025) are not meant to strictly prepare dedicated cell types, or in other words can rotate cell types. For instance, the chambers can alternate being used for RBC and WBC preparation. In this manner, once the samples in the chambers are prepped an image, a cleaning cycle can be utilized to clean the chambers before receiving a subsequent blood sample (e.g., a chamber can first be configured to prepare WBC's for a certain amount of same preparation runs, then RBC's for a certain amount of sample preparation runs—for instance 1 WBC preparation followed by 1 RBC preparation, or 2 WBC preparations followed by 1 RBC preparation followed by 2 more WBC preparations, etc). A cleaning reagent, such as diluent or cleaner, can be used between sample runs to eliminate carryover. Even in circumstances where a particular chamber is solely used for a particular cell type (e.g., 4020 used solely as a WBC chamber), there can be a cleaning step run after a sample is prepared and imaged in order to eliminate carryover.

Other embodiments can still utilize multiple stains as part of the preparation process. For instance, a first stain configured to stain white blood cells in the manner described herein, and a second stain configured to stain at least one of platelets or reticulocytes. These staining compositions can be used uniquely in various workflows. For instance, a first chamber can be used to prepare a white blood cell sample that comprises receiving at least a WBC stain and lyse reagent, while a second chamber can be used to prepare a platelet sample—this chamber would receive at least a platelet reagent-different than the WBC stain and lyse reagent.

Please note, though the term White blood cell (WBC) chamber and Red blood cell (RBC) chamber is used to denote the sample preparation chambers for imaging, the samples imaged as a result of the preparation process can allow for biological imaging of a plurality of cell types. For instance, the WBC chambers utilize a lyse to eliminate red blood cells, however the lyse may still retain platelets and reticulocytes, so the sample prepared in the WBC chamber can still image at least white blood cells, platelets, and reticulocytes—for instance. Similarly, the RBC chambers may receive a different preparation procedure than the WBC chambers (e.g., no lyse, or no stain/lyse combined reagent), but the sample prepared in the RBC chamber can still visualize a plurality of cell types, such as red blood cells- and one or more of white blood cells, platelets, and reticulocytes. Further information on how samples may be prepared for analysis in some embodiments, and in particular how stain may be applied in some cases is provided in U.S. patent application Ser. No. 18/224,947, the disclosure of which is incorporated herein by reference in its entirety.

### ADDITIONAL EXAMPLES

To further illustrate potential implementations and embodiments of the disclosed technology, exemplary systems and methods which could be practiced based on this disclosure are set forth below.

### Example 1A

A sample analysis system comprising: a) a flowcell; b) a fluidics system adapted to flow a portion of a sample through the flowcell; c) an image capture device configured to capture a plurality of images of blood cells as the blood cells pass through the flowcell; and d) one or more processors, the one or more processors programmed to perform acts comprising: i) analyzing the plurality of images to determine if a review indication applies to the plurality of images; ii) display an interface with the review indication and a description of the review indication; and iii) display the interface with at least one cell image corresponding to the review indication.

### Example 2A

The sample analysis system of example 1A, wherein the one or more processors are configured to determine that a user-defined review condition is satisfied; and, in response to determining that the user-defined review condition is satisfied, display the interface with the review indication.

### Example 3A

The sample analysis system of example 1A, wherein the review indication is at least one of: a high count indication, or a low count indication.

### Example 4A

The sample analysis system of example 1A, wherein the one or more processors are configured to determine that the review indication should be displayed based on satisfaction of a built in review condition.

### Example 5A

The sample analysis system of example 1A, wherein the one or more processors are configured to determine that the review indication should be displayed based on at least one of: a low confidence condition being satisfied, and a linearity condition not being satisfied.

### Example 6A

The sample analysis system of example 1A, wherein the one or more processors are programmed to determine that the review indication should be displayed based on detecting, in the plurality of images of blood cells, at least one of: platelet clumps or red blood cell clumps.

### Example 7A

The sample analysis system of example 1A, wherein the one or more processors are programmed to determine that the review indication should be displayed based on detecting, in the plurality of images of blood cells, at least one of: red blood cell fragments, sickle cells, dimorphic cells, large platelets, giant platelets, reticulated red blood cells, variant lymphocytes, or blast cells.

### Example 8A

The sample analysis system of example 1A, wherein the interface comprises a plurality of review indications and wherein the interface displays a description of each review indication and at least one cell image corresponding to each review indication.

### Example 9A

The sample analysis system of example 1A, further comprising a non-transitory computer readable medium having stored thereon a machine learning algorithm trained to analyze the images from the plurality of images to classify particles depicted in those images, wherein the one or more processors are programmed to determine that the review indication applies to the plurality of images based on confidence scores provided by the machine learning algorithm for classifications of particles depicted in the plurality of images.

### Example 10A

The sample analysis system of example 1A, further comprising a non-transitory computer readable medium storing a plurality of conditions for determining if corresponding review indications should be provided, wherein the plurality of conditions comprises a set of user defined conditions modifiable by users of the sample analysis system, and a set of built in conditions not modifiable by users of the sample analysis system.

### Example 11A

The sample analysis system of example 10A, wherein: a) each user defined from the set of user defined conditions is associated with a particular cell type; b) the set of user defined conditions comprise a first set of high and low thresholds for a particular cell type and a second set of high and low thresholds for the particular cell type; c) the one or more processors are programmed to: i) determine that a first review indication applies to the plurality of images when a count of the particular cell type is outside of the first set of high and low thresholds and contained within the second set of high and low thresholds; and ii) determine that a second review indication applies to the plurality of images when the count for the particular cell type is outside of the second set of high and low thresholds; and d) the first and second review indications are visually distinguishable from each other

### Example 12A

The sample analysis system of example 11A, wherein the first and second review indications have different colors.

### Example 13A

The sample analysis system of example 1A, wherein the at least one cell image corresponding to the review indication comprises a thumbnail cell image, and wherein the one or more processors are programmed to, in response to receiving a signal indicating user selection of the thumbnail cell image, display a full resolution image of a blood cell captured by the image capture device which corresponds to the thumbnail cell image.

### Example 14A

The sample analysis system of example 1A, wherein the at least one cell image corresponding to the review indication comprises a plurality of thumbnail cell images corresponding to the review indication, and wherein the plurality of thumbnail cell images corresponding to the review indication are sorted based on their respective contributions to the review indication.

### Example 15A

The sample analysis system of example 1A, wherein a) the one or more processors comprises: i) a first processor programmed to analyze the plurality of images to determine if the review indication applies to the plurality of images; and ii) a second processor programmed to display the interface; b) the second processor is comprised by an analyzer which also comprises the flowcell and the fluidics system; and c) the first processor is not comprised by the analyzer, and is separated from the second processor by, and in communication with the second processor via, a wide area network.

### Example 16A

A sample analysis method comprising: a) using a fluidics system, flowing a portion of a sample through a flowcell; b) using an image capture device, capturing a plurality of images of blood cells as the blood cells pass through the flowcell; c) using one or more processors, performing a set of acts comprising: i) analyzing the plurality of images to determine if a review indication applies to the plurality of images; ii) displaying an interface with the review indication and a description of the review indication; iii) displaying the interface with at least one cell image corresponding to the review indication.

### Example 17A

The sample analysis method of example 16A, wherein the method comprises determining that a user-defined review condition is satisfied; and wherein displaying the interface is performed in response to determining that the user-defined review condition is satisfied.

### Example 18A

The sample analysis method of example 16A, wherein the review indication is at least one of: a high count indication, or a low count indication.

### Example 19A

The sample analysis method of example 16A, wherein analyzing the plurality of images to determine if the review indication applies to the plurality of images comprises determining that the review indication should be displayed based on satisfaction of a built in review condition.

### Example 20A

The sample analysis method of example 16A, wherein analyzing the plurality of images to determine if the review indication applies to the plurality of images comprises determining that the review indication should be displayed based on at least one of: a low confidence condition being satisfied, and a linearity condition not being satisfied.

### Example 21A

The sample analysis method of example 16A, wherein analyzing the plurality of images to determine if the review indication applies to the plurality of images comprises determining that the review indication should be displayed based on detecting, in the plurality of images of blood cells, at least one of: platelet clumps or red blood cell clumps.

### Example 22A

The sample analysis method of example 16A, wherein analyzing the plurality of images to determine if the review indication applies to the plurality of images comprises determining that the review indication should be displayed based on detecting, in the plurality of images of blood cells, at least one of: red blood cell fragments, sickle cells, dimorphic cells, large platelets, giant platelets, reticulated red blood cells, variant lymphocytes, or blast cells.

### Example 23A

The sample analysis method of example 16A, wherein the interface comprises a plurality of review indications and wherein the interface displays a description of each review indication and at least one cell image corresponding to each review indication.

### Example 24A

The sample analysis method of example 16A, wherein analyzing the plurality of images to determine if the review indication applies to the plurality of images comprises: a) using a machine learning algorithm trained to analyze the images from the plurality of images to classify particles depicted in those images; and b) determining that the review indication applies to the plurality of images based on confidence scores provided by the machine learning algorithm for classifications of particles depicted in the plurality of image.

### Example 25A

The sample analysis method of example 16A, wherein analyzing the plurality of images to determine if the review indication applies to the plurality of images comprises retrieving, from a non-transitory computer readable medium, a plurality of conditions for determining if corresponding review indications should be provided, wherein the plurality of conditions comprises a set of user defined conditions modifiable by users of a sample analysis system, and a set of built in conditions not modifiable by users of the sample analysis system.

### Example 26A

The sample analysis method of example 25A, wherein: a) each user defined from the set of user defined conditions is associated with a particular cell type; b) the set of user defined conditions comprise a first set of high and low thresholds for a particular cell type and a second set of high and low thresholds for the particular cell type; c) the method comprises: i) determining whether a first review indication applies to the plurality of images based on whether a count of the particular cell type is outside of the first set of high and low thresholds and contained within the second set of high and low thresholds; and ii) determining whether a second review indication applies to the plurality of images based on whether the count for the particular cell type is outside of the second set of high and low thresholds; and d) the first and second review indications are visually distinguishable from each other.

### Example 27A

The sample analysis method of example 26A, wherein the first and second review indications have different colors.

### Example 28A

The sample analysis method of example 16A, wherein: a) the at least one cell image corresponding to the review indication comprises a thumbnail cell image; and b) the method comprises: i) receiving a signal indicating user selection of the thumbnail cell image; and ii) in response to receiving a signal indicating user selection of the thumbnail cell image, displaying a full resolution image of a blood cell captured by the image capture device which corresponds to the thumbnail cell image.

### Example 29A

The sample analysis method of example 16A, wherein the at least one cell image corresponding to the review indication comprises a plurality of thumbnail cell images corresponding to the review indication, and wherein the method comprises sorting the plurality of thumbnail cell images corresponding to the review indication based on their respective contributions to the review indication.

### Example 30A

The sample analysis method of example 16A, wherein: a) the one or more processors comprises: i) a first processor programmed to analyze the plurality of images to determine if the review indication applies to the plurality of images; and ii) a second processor programmed to display the interface; b) the second processor is comprised by an analyzer which also comprises the flowcell and the fluidics system; and c) the first processor is not comprised by the analyzer, and is separated from the second processor by, and in communication with the second processor via, a wide area network.

### Example 31A

A method of using a biological analyzer comprising: a) using a fluidics system, flowing a portion of a sample through a flowcell; b) using an image capture device, capturing a plurality of images of blood cells as the blood cells pass through the flowcell; and c) viewing a review indication associated with the sample; and d) reviewing the review indication by accessing data corresponding to the review indication through a user interface.

### Example 32A

The method of example 31A, wherein: a) the review indication associated with the sample is associated with at least a portion of the plurality of images; and b) reviewing the review indication by accessing data corresponding to the review indication through the user interface is performed by reviewing at least a subset of the at least the portion.

### Example 33A

The method of example 32A, wherein: a) the method comprises: i) viewing a set of thumbnails of cell images having a type corresponding to the review indication; and ii) selecting a thumbnail from the set of thumbnails; and b) reviewing the subset of the at least the portion of the plurality of images comprises viewing a full resolution image corresponding to the selected thumbnail.

### Example 34A

The method of example 33A, wherein the method comprises selecting a sorting criteria for the set of thumbnails of cell images having the type corresponding to the review indication.

### Example 35A

The method of example 32A, wherein: a) accessing data corresponding to the review indication comprises reviewing a message indicating an abnormal measurement derived from the plurality of images of blood cells; and b) the method comprises confirming whether the abnormal measurement derived from the plurality of images is correct based on reviewing additional information corresponding to the abnormal result.

### Example 36A

The method of example 35A, wherein confirming whether the abnormal measurement derived from the plurality of images is correct based on reviewing additional information corresponding to the abnormal result comprises viewing one or more full resolution images from the plurality of images of blood cells.

### Example 37A

The method of example 35A, wherein confirming whether the abnormal measurement derived from the plurality of images is correct based on reviewing additional information corresponding to the abnormal result comprises viewing a result derived by a non-imaging measurement system.

### Example 38A

The method of example 37A, wherein the abnormal measurement derived from the plurality of images is a count for a type of cells, and wherein the result derived by the non-imaging measurement system is a count for the same type of cells.

### Example 39A

The method of example 38A, wherein the method comprises, based on confirming whether the abnormal measurement derived from the plurality of images is correct, determining whether to run a count for the same type of cells using a new portion of the sample.

### Example 40A

The method of example 31A, wherein the method further comprises defining, for at least one cell type from a plurality of cell types, a review condition for that cell type.

### Example 41A

The method of example 40A, wherein the review condition comprises a plurality of sets of thresholds, wherein each set of thresholds comprises a high threshold and a low threshold.

### Example 42A

The method of example 31A, wherein the method comprises determining, based on accessing the data corresponding to the review indication through the user interface, that an additional analysis should be performed on the sample.

### Example 43A

The method of example 42A, wherein: a) the additional analysis comprises capturing images of reticulated red blood cells in the sample; b) the method comprises the user accessing one or more of the images of reticulated blood cells; and c) accessing the data corresponding to the review indication through the user interface comprises accessing a reticulated red blood cell count for the sample.

### Example 44A

The method of example 42A, wherein: a) accessing data corresponding to the review indication comprises reviewing a message indicating a count for the sample based on the portion of the sample exceeds a maximum approved count; and b) the additional analysis comprises re-determining the count using a new portion of the sample.

### Example 45A

The method of example 44A, wherein the method comprises diluting the new portion of the sample to a higher dilution level than a dilution level used for the portion of the sample which formed a basis of the count which exceeded the maximum approved count.

### Example 1B

A sample analysis system comprising: a) a fluidics system adapted to: i) flow a first portion of a blood sample through a first module, the first module being a flow imaging module comprising a flowcell and an image capture device configured to capture a plurality of images of cells of the first portion of the blood sample; and ii) flow a second portion of the blood sample through a second module, the second module configured to test for one or more numerical parameters of cells of the second portion of the blood sample; and b) one or more processors programmed to: i) determine the one or more numerical parameters of cells of the second portion of the blood sample; and ii) present a computing interface comprising the plurality of images of the cells of the first portion of the blood sample and the one or more numerical parameters of the cells of the second portion of the blood sample.

### Example 2B

The sample analysis system of example 1B, wherein the sample analysis system further comprises an aliquoter configured to separate the blood sample into a plurality of aliquots, wherein the first portion is a first aliquot from the plurality of aliquots, and the second portion is a second aliquot from the plurality of aliquots.

### Example 3B

The sample analysis system of example 1B, wherein the sample analysis system is adapted to: a) receive the blood sample in a container bearing a barcode; b) read the barcode; and c) determine one or more tests for the blood sample based on the barcode.

### Example 4B

The sample analysis system of example 1B, wherein: a) the fluidics system is adapted to flow a first subportion of the first portion of the blood sample through the flow imaging module for red blood cell (RBC) imaging in the first flowcell; and b) the fluidics system is adapted to flow a second subportion of the first portion of the blood sample through the flow imaging module for white blood cell (WBC) imaging, the second subportion being treated with a stain composition.

### Example 5B

The sample analysis system of example 1B, wherein the second module comprises an impedance analyzer.

### Example 6B

The sample analysis system of example 1B, wherein the second module comprises a fluorescence analyzer.

### Example 7B

The sample analysis system of example 1B, wherein the numerical parameter is selected from a mean corpuscular volume, a cell count, and a hemoglobin concentration.

### Example 8B

The sample analysis system of example 1B, wherein the plurality of images includes images of a first cell type and images of a second cell type.

### Example 9B

The sample analysis system of example 1B, wherein the plurality of cells includes a first cell type, and wherein the computing interface is configured to allow a user to select the first cell type, and display images of the first cell type in response.

### Example 10B

The sample analysis system of example 1B, wherein the plurality of cells includes a first cell type and a second cell type, and wherein the computing interface is configured to allow a user to select a first cell type and a second cell type, and display images of the first cell type and the second cell type in response.

### Example 11B

The sample analysis system of example 1B, wherein the one or more processors are further programmed to derive numerical data from the plurality of images and present the numerical data on the computing interface.

### Example 12B

The sample analysis system of example 1B, wherein the second module is further configured to test for one or more numerical parameters of a first cell type, and to test for one or more numerical parameters of a second type.

### Example 13B

The sample analysis system of example 1B, wherein the second module is configured to determine more than one parameter for a first cell type.

### Example 14B

The sample analysis system of example 1B, wherein the computing interface is configured to provide the plurality of images of the cells of the first portion of the blood sample and the one or more numerical parameters of the second portion of the blood sample on a single screen.

### Example 15B

The sample analysis system of example 1B, wherein: a) the one or more processors comprises: i) a first processor programmed to determine the one or more parameters of cells of the second portion of the blood sample; and ii) a second processor programmed to present the computing interface comprising the plurality of images of the cells of the first portion of the blood sample and the one or more numerical parameters of the cells of the second portion of the blood sample; b) the second processor is comprised by an analyzer which also comprises the fluidics system; and c) the first processor is not comprised by the analyzer, and is separated from the second processor by, and in communication with the second processor via, a wide area network.

### Example 16B

A sample analysis method comprising: a) using a fluidics system: i) flowing a first portion of a blood sample through a first module, the first module being a flow imaging module comprising a flowcell and an image capture device configured to capture a plurality of images of cells of the first portion of the blood sample; and ii) flowing a second portion of the blood sample through a second module, the second module configured to test for one or more numerical parameters of cells of the second portion of the blood sample; and b) using one or more processors: i) determining the one or more numerical parameters of cells of the second portion of the blood sample; and ii) presenting a computing interface comprising the plurality of images of the cells of the first portion of the blood sample and the one or more numerical parameters of the cells of the second portion of the blood sample.

### Example 17B

The sample analysis method of example 16B, wherein the method comprises separating the blood sample into a plurality of aliquots using an aliquoter, wherein the first portion is a first aliquot from the plurality of aliquots, and the second portion is a second aliquot from the plurality of aliquots.

### Example 18B

The sample analysis method of example 16B, wherein the method comprises: a) receiving the blood sample in a container bearing a barcode; b) reading the barcode; and c) determining one or more tests for the blood sample based on the barcode.

### Example 19B

The sample analysis method of example 16B, wherein: a) the fluidics system is adapted to flow a first subportion of the first portion of the blood sample through the flow imaging module for red blood cell (RBC) imaging in the first flowcell; and b) the fluidics system is adapted to flow a second subportion of the first portion of the blood sample through the flow imaging module for white blood cell (WBC) imaging, the second subportion being treated with a stain composition.

### Example 20B

The sample analysis method of example 16B, wherein the second module comprises an impedance analyzer.

### Example 21B

The sample analysis method of example 16B, wherein the second module comprises a fluorescence analyzer.

### Example 22B

The sample analysis method of example 16B, wherein the numerical parameter is selected from a mean corpuscular volume, a cell count, and a hemoglobin concentration.

### Example 23B

The sample analysis method of example 16B, wherein the plurality of images includes images of a first cell type and images of a second cell type.

### Example 24B

The sample analysis method of example 16B, wherein the plurality of cells includes a first cell type, and wherein the computing interface is configured to allow a user to select the first cell type, and display images of the first cell type in response.

### Example 25B

The sample analysis method of example 16B, wherein the plurality of cells includes a first cell type and a second cell type, and wherein the computing interface is configured to allow a user to select a first cell type and a second cell type, and display images of the first cell type and the second cell type in response.

### Example 26B

The sample analysis method of example 16B, wherein the one or more processors are further programmed to derive numerical data from the plurality of images and present the numerical data on the computing interface.

### Example 27B

The sample analysis method of example 16B, wherein the second module is further configured to test for one or more numerical parameters of a first cell type, and to test for one or more numerical parameters of a second type.

### Example 28B

The sample analysis method of example 16B, wherein the second module is configured to determine more than one parameter for a first cell type.

### Example 29B

The sample analysis method of example 16B, wherein the computing interface is configured to provide the plurality of images of the cells of the first portion of the blood sample and the one or more numerical parameters of the second portion of the blood sample on a single screen.

### Example 30B

The sample analysis method of example 16B, wherein: a) the one or more processors comprises: i) a first processor programmed to determine the one or more parameters of cells of the second portion of the blood sample; and ii) a second processor programmed to present the computing interface comprising the plurality of images of the cells of the first portion of the blood sample and the one or more numerical parameters of the cells of the second portion of the blood sample; b) the second processor is comprised by an analyzer which also comprises the fluidics system; and c) the first processor is not comprised by the analyzer, and is separated from the second processor by, and in communication with the second processor via, a wide area network.

### Example 1C

A sample analysis system comprising: a) a fluidics system adapted to: i) flow a first portion of a blood sample through a first module, the first module being a flow imaging module comprising a flowcell and an image capture device configured to capture a plurality of images of cells of a first type; and ii) flow a second portion of the blood sample through a second module, the second module configured to test for one or more numerical parameters of cells of the first type; b) one or more processors programmed to: i) determine one or more image-based numerical values of the first cell type from the plurality of images from the first module; ii) determine the one or more numerical parameters of the first cell type from the second module; iii) present a computing interface comprising the one or more image-based numerical values of the first cell type, and the one or more numerical parameters of the first cell type.

### Example 2C

The sample analysis system of example 1C, wherein the first cell type is a red blood cell or a platelet.

### Example 3C

The sample analysis system of example 1C wherein the fluidics system is adapted to capture a plurality of images of cells of a second type and test for one or more numerical parameters of cells of a second type, and wherein the one or more processors are programmed to determine one or more image-based numerical values of the second cell type from the plurality of images from the first module, determine the one or more numerical parameters of the second cell type from the second module, and present a computing interface comprising the one or more image-based numerical values of the first cell type and the one or more numerical parameters of the second cell type.

### Example 4C

The sample analysis system of example 3C, wherein the first cell type is a red blood cell and the second cell type is a platelet.

### Example 5C

The sample analysis system of example 1C, wherein the first cell type is a red blood cell, the one or more image-based parameters comprise a red blood cell count, and the one or more numerical parameters comprise a mean corpuscular volume.

### Example 6C

The sample analysis system of example 1C, wherein the second module comprises an impedance analyzer.

### Example 7C

The sample analysis system of example 1C, wherein the second module comprises a fluorescence analyzer.

### Example 8C

The sample analysis system of example 1C, wherein the second module comprises a spectrophotometric analyzer.

### Example 9C

The sample analysis system of example 1C, wherein the first cell type is a platelet, the one or more image-based parameters comprise a platelet count, and the one or more numerical parameters comprise a platelet volume.

### Example 10C

The sample analysis system of example 1C, wherein the sample analysis system comprises an identification reader configured to read sample identifiers, and a controller programmed to determine parameters to determine values for based on data from the identification reader.

### Example 11C

The sample analysis system of example 10, wherein the sample analysis system comprises an aliquoter configured to separate samples into aliquots, and wherein the controller is programmed to cause the fluidics system to control the flow of aliquots based on the parameters to determine values for.

### Example 12C

The sample analysis system of example 1C, wherein the one or more processors are programmed to present the computing interface comprising the one or more image-based numerical values of the first cell type, and the one or more numerical parameters of the first cell type on a single screen.

### Example 13C

The sample analysis system of example 1C, wherein the first cell type is a red blood cell, the one or more image-based parameters comprise a red blood cell count, and the one or more numerical parameters comprise a hemoglobin measurement.

### Example 14C

The sample analysis system of example 1C, wherein the computing interface is configured to have a user select the first cell type and then display the plurality of images of the first cell type in response.

### Example 15C

The sample analysis system of example 1C, wherein: a) the one or more processors comprises: i) a first processor programmed to determine the one or more image-based numerical values of the first cell type from the plurality of images from the first module; and ii) a second processor programmed to present the computing interface comprising the one or more image-based numerical values of the first cell type, and the one or more numerical parameters of the first cell type; b) the second processor is comprised by an analyzer which also comprises the fluidics system; c) the first processor is not comprised by the analyzer, and is separated from the second processor by, and in communication with the second processor via, a wide area network.

### Example 16C

A sample analysis method comprising: a) using a fluidics system: i) flow a first portion of a blood sample through a first module, the first module being a flow imaging module comprising a flowcell and an image capture device configured to capture a plurality of images of cells of a first type; and ii) flow a second portion of the blood sample through a second module, the second module configured to test for one or more numerical parameters of cells of the first type; and b) using one or more processors: i) determine one or more image-based numerical values of the first cell type from the plurality of images from the first module; ii) determine the one or more numerical parameters of the first cell type from the second module; iii) present a computing interface comprising the one or more image-based numerical values of the first cell type, and the one or more numerical parameters of the first cell type.

### Example 17C

The sample analysis method of example 16C, wherein the first cell type is a red blood cell or a platelet.

### Example 18C

The sample analysis method of example 16C wherein the fluidics system is adapted to capture a plurality of images of cells of a second type and test for one or more numerical parameters of cells of a second type, and wherein the one or more processors are programmed to determine one or more image-based numerical values of the second cell type from the plurality of images from the first module, determine the one or more numerical parameters of the second cell type from the second module, and present a computing interface comprising the one or more image-based numerical values of the first cell type and the one or more numerical parameters of the second cell type.

### Example 19C

The sample analysis method of example 18C, wherein the first cell type is a red blood cell and the second cell type is a platelet.

### Example 20C

The sample analysis method of example 16C, wherein the first cell type is a red blood cell, the one or more image-based parameters comprise a red blood cell count, and the one or more numerical parameters comprise a mean corpuscular volume.

### Example 21C

The sample analysis method of example 16C, wherein the second module comprises an impedance analyzer.

### Example 22C

The sample analysis method of example 16C, wherein the second module comprises a fluorescence analyzer.

### Example 23C

The sample analysis method of example 16C, wherein the second module comprises a spectrophotometric analyzer.

### Example 24C

The sample analysis method of example 16C, wherein the first cell type is a platelet, the one or more image-based parameters comprise a platelet count, and the one or more numerical parameters comprise a platelet volume.

### Example 25C

The sample analysis method of example 16C, wherein the sample analysis system comprises an identification reader configured to read sample identifiers, and a controller programmed to determine parameters to determine values for based on data from the identification reader.

### Example 26C

The sample analysis method of example 25C, wherein the sample analysis system comprises an aliquoter configured to separate samples into aliquots, and wherein the controller is programmed to cause the fluidics system to control the flow of aliquots based on the parameters to determine values for.

### Example 27C

The sample analysis method of example 16C, wherein the one or more processors are programmed to present the computing interface comprising the one or more image-based numerical values of the first cell type, and the one or more numerical parameters of the first cell type on a single screen.

### Example 28C

The sample analysis method of example 16C, wherein the first cell type is a red blood cell, the one or more image-based parameters comprise a red blood cell count, and the one or more numerical parameters comprise a hemoglobin measurement.

### Example 29C

The sample analysis method of example 16C, wherein the computing interface is configured to have a user select the first cell type and then display the plurality of images of the first cell type in response.

### Example 30C

The sample analysis method of example 16C, wherein: a) the one or more processors comprises: i) a first processor programmed to determine the one or more image-based numerical values of the first cell type from the plurality of images from the first module; and ii) a second processor programmed to present the computing interface comprising the one or more image-based numerical values of the first cell type, and the one or more numerical parameters of the first cell type; b) the second processor is comprised by an analyzer which also comprises the fluidics system; c) the first processor is not comprised by the analyzer, and is separated from the second processor by, and in communication with the second processor via, a wide area network.

### Interpretation

It should be understood that, in the above examples and the claims, a statement that something is “based on” something else should be understood to mean that it is determined at least in part by the thing that it is indicated as being based on. To indicate that something must be completely determined based on something else, it is described as being “based EXCLUSIVELY on” whatever it must be completely determined by.

It should be understood that a statement that “one or more” or “at least one” of a type of item have a characteristic indicates that the items in the indicated group collectively have the characteristic. To indicate that each item in a group has a characteristic, the phrase “each of” will be used with the group identifier (e.g., “one or more” or “at least one”).

It should be understood that, in the claims, “set” should be understood as referring to one or more thing of similar nature, design or function.

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A sample analysis system comprising:
a) a fluidics system adapted to:
i) flow a first portion of a blood sample through a first module, the first module being a flow imaging module comprising a flowcell and an image capture device configured to capture a plurality of images of cells of a first cell type; and
ii) flow a second portion of the blood sample through a second module, the second module configured to test for one or more numerical parameters of cells of the first cell type;
and
b) one or more processors programmed to:
i) determine one or more image-based numerical values of the first cell type from the plurality of images from the first module;
ii) determine the one or more numerical parameters of the first cell type from the second module;
iii) analyze the plurality of images from the first module and determine one or more labels for cells of the first cell type using a machine learning model; and
iv) present a computing interface comprising the one or more image-based numerical values of the first cell type, the one or more numerical parameters of the first cell type, and at least one of the determined one or more labels for cells of the first cell type.

2. The sample analysis system of claim 1, wherein the first cell type is a red blood cell or a platelet.

3. The sample analysis system of claim 1 wherein the fluidics system is adapted to capture a plurality of images of cells of a second cell type and test for one or more numerical parameters of cells of the second cell type, and wherein the one or more processors are programmed to determine one or more image-based numerical values of the second cell type from the plurality of images from the first module, determine the one or more numerical parameters of the second cell type from the second module, and present a computing interface comprising the one or more image-based numerical values of the first cell type and the one or more numerical parameters of the second cell type.

4. The sample analysis system of claim 3, wherein the first cell type is a red blood cell and the second cell type is a platelet.

5. The sample analysis system of claim 1, wherein the first cell type is a red blood cell, the one or more image-based parameters comprise a red blood cell count, and the one or more numerical parameters comprise a mean corpuscular volume.

6. The sample analysis system of claim 1, wherein the second module comprises an impedance analyzer.

7. The sample analysis system of claim 1, wherein the second module comprises a fluorescence analyzer.

8. The sample analysis system of claim 1, wherein the second module comprises a spectrophotometric analyzer.

9. The sample analysis system of claim 1, wherein the first cell type is a platelet, the one or more image-based parameters comprise a platelet count, and the one or more numerical parameters comprise a platelet volume.

10. The sample analysis system of claim 1, wherein the sample analysis system comprises an identification reader configured to read sample identifiers, and a controller programmed to determine parameters to determine values for based on data from the identification reader.

11. The sample analysis system of claim 10, wherein the sample analysis system comprises an aliquoter configured to separate samples into aliquots, and wherein the controller is programmed to cause the fluidics system to control the flow of aliquots based on the parameters to determine values for.

12. The sample analysis system of claim 1, wherein the one or more processors are programmed to present the computing interface comprising the one or more image-based numerical values of the first cell type, and the one or more numerical parameters of the first cell type on a single screen.

13. The sample analysis system of claim 1, wherein the first cell type is a red blood cell, the one or more image-based parameters comprise a red blood cell count, and the one or more numerical parameters comprise a hemoglobin measurement.

14. The sample analysis system of claim 1, wherein the computing interface is configured to have a user select the first cell type and then display the plurality of images of the first cell type in response.

15. The sample analysis system of claim 1, wherein:
a) the one or more processors comprises:
i) a first processor programmed to determine the one or more image-based numerical values of the first cell type from the plurality of images from the first module; and
ii) a second processor programmed to present the computing interface comprising the one or more image-based numerical values of the first cell type, and the one or more numerical parameters of the first cell type;
b) the second processor is comprised by an analyzer which also comprises the fluidics system;
c) the first processor is not comprised by the analyzer, and is separated from the second processor by, and in communication with the second processor via, a wide area network.

16. A sample analysis method comprising:
a) using a fluidics system:
i) flow a first portion of a blood sample through a first module, the first module being a flow imaging module comprising a flowcell and an image capture device configured to capture a plurality of images of cells of a first cell type; and
ii) flow a second portion of the blood sample through a second module, the second module configured to test for one or more numerical parameters of cells of the first cell type;
and
b) using one or more processors:
i) determine one or more image-based numerical values of the first cell type from the plurality of images from the first module;
ii) determine the one or more numerical parameters of the first cell type from the second module;
iii) analyze the plurality of images from the first module and determine one or more labels for cells of the first cell type by using a machine learning model; and
iv) present a computing interface comprising the one or more image-based numerical values of the first cell type, the one or more numerical parameters of the first cell type, and at least one of the determined one or more labels for cells of the first cell type.

17-24. (canceled)

25. The sample analysis method of claim 16, wherein the sample analysis system comprises an identification reader configured to read sample identifiers, and a controller programmed to determine parameters to determine values for based on data from the identification reader.

26. (canceled)

27. The sample analysis method of claim 16, wherein the one or more processors are programmed to present the computing interface comprising the one or more image-based numerical values of the first cell type, and the one or more numerical parameters of the first cell type on a single screen.

28. (canceled)

29. The sample analysis method of claim 16, wherein the computing interface is configured to have a user select the first cell type and then display the plurality of images of the first cell type in response.

30. (canceled)

31. A sample analysis system comprising:
one or more processors; and
one or more non-transitory computer readable mediums storing instructions to, when executed, perform a set of acts comprising:
flowing a first portion of a blood sample through a first module, the first module being a flow imaging module comprising a flowcell and an image capture device configured to capture a plurality of images of cells of a first cell type;
flowing a second portion of the blood sample through a second module, the second module configured to test for one or more numerical parameters of cells of the first cell type;
determining one or more image-based numerical values of the first cell type from the plurality of images from the first module;
determining the one or more numerical parameters of the first cell type from the second module;
analyzing the plurality of images from the first module and determine one or more labels for cells of the first cell type by using a machine learning model; and
presenting a computing interface comprising the one or more image-based numerical values of the first cell type, and the one or more numerical parameters of the first cell type, and at least one of the determined one or more labels for cells of the first cell type.
